(19) United States
(12) Patent Application Publication
(10) Pub. No.: US 2021/0358566 A1
(43) Pub. Date: Nov. 18, 2021

(54) RESOLUTION INDICES FOR DETECTING HETEROGENEITY IN DATA AND METHODS OF USE THEREOF

(71) Applicant: Becton, Dickinson and Company, Franklin Lakes, NJ (US)

(72) Inventor: Ian James Taylor, Talent, OR (US)

(21) Appl. No.: 17/313,449

(22) Filed: May 6, 2021

Related U.S. Application Data
(60) Provisional application No. 63/026,327, filed on May 18, 2020.

(51) Int. Cl. G16B 30/00; G01N 15/14

(52) U.S. Cl. CPC G16B 30/00; G01N 15/1429

(57) ABSTRACT

Methods for detecting heterogeneity in data (e.g., flow cytometer data, nucleic acid sequence data) are provided. In some instances, methods include generating one or more population clusters based on the determined parameters of the analytes (e.g., cells, particles, nucleic acids) in a biological sample sample. In embodiments, methods include calculating a resolution index by computing a ratio between measures of variability and separation distance for any given number of pairs of first and second populations of data. Where desired, methods also include maximizing the resolution between populations of data by computing a resolution score that accounts for the sum of resolution indices, the number of populations, the number of parameters and the number of cells. Systems and computer-readable media for determining heterogeneity between populations of data and, where desired, maximizing resolution between populations of data, are also provided.

### CROSS-REFERENCE

Pursuant to 35 U.S.C. § 119 (e), this application claims priority to the filing date of U.S. Provisional Patent Application Ser. No. 631026,327 filed May 18, 2020, the disclosure of which application is incorporated herein by reference in its entirety.

### INTRODUCTION

The characterization of analytes in biological samples has become an important part of medical diagnoses and assessments of overall health and wellness of a patient, Methods of analyzing biological samples, such as flow cytometry and nucleic acid sequencing, have broad applications in the field of biological research,

Flow cytometry is a technique used to characterize and often times sort biological material, such as cells of a blood sample or particles of interest in another type of biological or chemical sample. A flow cytometer typically includes a sample reservoir for receiving a fluid sample, such as a blood sample, and a sheath reservoir containing a sheath fluid. The flow cytometer transports the particles (including cells) in the fluid sample as a cell stream to a flow cell, while also directing the sheath fluid to the flow cell. To characterize the components of the flow stream, the flow stream is irradiated with light. Variations in the materials in the flow stream, such as morphologies or the presence of fluorescent labels, may cause variations in the observed light and these variations allow for characterization and separation. For example, particles, such as molecules, analyte-bound beads, or individual cells, in a fluid suspension are passed by a detection region in which the particles are exposed to an excitation light, typically from one or more lasers, and the light scattering and fluorescence properties of the particles are measured. Particles or components thereof typically are labeled with fluorescent dyes to facilitate detection. A multiplicity of different particles or components may be simultaneously detected by using spectrally distinct fluorescent dyes to label the different particles or components. In some implementations, a multiplicity of photodetectors, one for each of the scatter parameters to be measured, and one or more for each of the distinct dyes to be detected are included in the analyzer. For example, some embodiments include spectral configurations where more than one sensor or detector is used per dye. The data obtained comprise the signals measured for each of the light scatter detectors and the fluorescence emissions.

Particle analyzers may further comprise means for recording the measured data and analyzing the data. For example, data storage and analysis may be carried out using a computer connected to the detection electronics. For example, the data can be stored in tabular form, where each row corresponds to data for one particle, and the columns correspond to each of the measured features. The use of standard file formats, such as an “FCS” file format, for storing data from a particle analyzer facilitates analyzing data using separate programs and/or machines. Using current analysis methods, the data typically are displayed in 1-dimensional histograms or 2-dimensional (2D) plots for ease of visualization, but other methods may be used to visualize multidimensional data.

The parameters measured using, for example, a flow cytometer typically include light at the excitation wavelength scattered by the particle in a narrow angle along a mostly forward direction, referred to as forward scatter (FSC), the excitation light that is scattered by the particle in an orthogonal direction to the excitation laser, referred to as side scatter (SSC), and the light emitted from fluorescent molecules in one or more detectors that measure signal over a range of spectral wavelengths, or by the fluorescent dye that is primarily detected in that specific detector or array of detectors. Different cell types can be identified by their light scatter characteristics and fluorescence emissions resulting from labeling various cell proteins or other constituents with fluorescent dye-labeled antibodies or other fluorescent probes.

Both flow and scanning cytometers are commercially available from, for example, BD Biosciences (San Jose, Calif.). Flow cytometry is described in, for example, Landy et al. (eds.), Clinical Flow Cytometry, Annals of the New York Academy of Sciences Volume 677 (1993); Bauer et al. (eds.), Clinical Flow Cytometry: Principles and Applications, Williams & Wilkins (1993); Ormerod (ed.), Flow Cytometry: A Practical Approach, Oxford Univ. Press (1994); Jaroszeski et al. (eds.), Flow Cytometry Protocols, Methods in Molecular Biology No. 91, Humana Press (1997); and Practical Shapiro, Flow Cytometry, 4th ed., Wiley-Liss (2003); all incorporated herein by reference. Fluorescence imaging microscopy is described in, for example, Pawley (ed.), Handbook of Biological Confocal Microscopy, 2nd Edition, Plenum Press (1989), incorporated herein by reference.

Nucleic acid sequencing methods include the Sanger “dideoxy” method which relies upon the use of dideoxyribonucleoside triphosphates as chain terminators. The Sanger method has been adapted for use in automated sequencing with the use of chain terminators incorporating fluorescent labels. Other methods include “next-generation” sequencing methods, including those based on successive cycles of incorporation of fluorescently labeled nucleic acid analogues. In such “sequencing by synthesis” or “cycle sequencing” methods the identity of the added base is determined after each nucleotide addition by detecting the fluorescent label. Other next-generation sequencing methods include those based on the detection of hydrogen ions that are released during the polymerization of DNA. A microwell containing a template DNA strand to be sequenced is flooded with a single species of deoxyribonucleotide triphosphate (dNTP). If the introduced dNTP is complementary to the leading template nucleotide, it is incorporated into the growing complementary strand. This incorporation causes the release of a hydrogen ion that triggers an ISFET ion sensor, which indicates that a reaction has occurred. If homopolymer repeats are present in the template sequence, multiple dNTP molecules will be incorporated in a single cycle. This leads to a corresponding number of released hydrogens and a proportionally higher electronic signal.

The data obtained from an analysis of cells (or other particles) by flow cytometry or nucleic acid sequencing are multidimensional when each cell corresponds to a point in a multidimensional space defined by the parameters measured. Populations of cells or particles are identified as clusters of points in the data space. The identification of clusters and, thereby, populations can be carried out manually by drawing a gate around a population displayed in one or more 2-dimensional plots, referred to as “scatter plots” or “dot plots,” of the data. Alternatively, population clusters can be identified, and gates that define the limits of the populations, can be determined automatically. Examples of methods for automated gating have been described in, for example, U.S. Pat. Nos. 4,845,653; 5,627,040; 5,739,000; 5,795,727; 5,962,238; 6,014,904; and 6,944,338; and U.S. Pat. Pub. No. 2012/0245889, each incorporated herein by reference.

However, distinguishing different groups of analytes (e.g., particles, cells, nucleic acids) into discrete populations often presents a challenge because it may not be obvious that two populations are sufficiently distinct such that they constitute separate clusters. Conventional methods for distinguishing populations of data often include computing a “stain index”, a measure of separation used in the field of data analysis for assessing the signal to noise ratio for two populations in a univariate parameter. In other words, the stain index provides a measure of separation between a “positive” population of data (i.e., cells encompassed by the population are positive for a given parameter) and a “negative” population of flow cytometer data (i.e., cells encompassed by the population are negative for a given parameter). The stain index is computed by dividing the separation between the positive and negative populations by two times the standard deviation of the negative population. For example, FIG. 1 provides a sample stain index calculation measuring the separation between a population of flow cytometer data that is positive for CD14 and a population of flow cytometer data that is negative for CD14. However, as noted above and demonstrated in FIG. 1, the stain index only accounts for the variance of the negative population of data. As a result, the stain index is biased toward the variance of the negative peak and is insensitive to variance inherent to the positive peak. This is a problem for, e.g., CyTOF and scRNA sequencing datasets in which negative populations tend to be very narrow.

### SUMMARY

Aspects of the invention include detecting heterogeneity in data. In some embodiments, the data is flow cytometer data (e.g., data produced by a flow cytometer). In other embodiments, the data is nucleic acid sequence data (e.g., data produced by a nucleic acid sequencing platform). In some embodiments, methods include generating one or more population clusters of data (e.g., flow cytometer data, nucleic acid sequence data) based on the determined parameters of analytes (e.g., cells, particles, nucleic acids) in the sample. In these embodiments, methods include receiving data, calculating parameters of each analyte, and clustering together analytes based on the calculated parameters. In certain instances, detecting heterogeneity in the data includes calculating a resolution index for any given number of adjacent first and second populations of data. In some embodiments, the first population of data is positive for a given parameter, and the second population of data is negative for that parameter. In embodiments, calculating a resolution index includes obtaining measures of variability (e.g., mean, standard deviation) from the first and second populations of data, determining a separation distance between the first and second populations of data, and computing a ratio between the respective measures of variability for the first and second populations of data and the separation distance. The resultant resolution index may be used to provide a quantification of the separation between populations of data and, if desired, maximize the resolution between different populations. In some embodiments, methods include generating an image (e.g., heatmap, scatterplot) to depict heterogeneity as determined by one or more measures of separation between populations of data (e.g., resolution index, Hartigan's dip statistic). In embodiments where it is desirable to maximize resolution between populations of data, aspects of the invention include computing a resolution score that accounts for the sum of resolution indices, the number of populations, the number of parameters and the number of cells. In some embodiments, a resolution score is calculated for each value of n (i.e., number of populations) such that there is a resolution score associated with each possible number of populations. i.e., to determine an optimal number and arrangement of population clusters that maximizes the resolution of the data. In addition, embodiments of the invention include reducing the dimensionality of the data by subjecting the data to a dimensionality reduction algorithm that has been selected because it produces population clusters that possess a higher resolution score than other dimensionality reduction algorithms.

Aspects of the invention also include an apparatus configured to produce data by analyzing a biological sample. In some instances, the apparatus is a flow cytometer configured to produce flow cytometer data. Flow cytometers according to embodiments of the invention also include detectors configured to detect particle-modulated light (e.g., scattered light produced by a particle passing through a laser at an interrogation point in a flow cell, e.g., fluorescent light, light emitted by a particle after passing through a laser at an interrogation point in a flow cell, etc.). For example, flow cytometers of interest may include one or more forward and/or side scatter detectors configured to detect side scattered light from the flow cell, as well as one or more fluorescent light detectors configured to detect fluorescent light from the flow cell. In other embodiments, the apparatus is a nucleic acid sequencing platform configured to produce nucleic acid sequence data. Nucleic acid sequencing platforms according to embodiments of the invention may be any sequencing system of interest, including a Sanger sequencing system, a next generation sequencing (NGS) system, or the like. In certain aspects the sequencing system is an NGS system.

Systems of interest also include logic, e.g., software and/or hardware, such as a processor having memory operably coupled to the processor wherein the memory includes instructions stored thereon, which when executed by the processor, cause the processor to detect heterogeneity in data (e.g., flow cytometry data, nucleic acid sequence data) and, where desired, maximize the resolution between populations of data. In embodiments, the processor is configured to classify data according to one or more different parameters, detect heterogeneity in the data by calculating a resolution index for any given number of adjacent first and second populations of data, calculate Hartigan's dip statistic for each population of data and generate an image composed of heatmaps or plots. Where desired, the processor includes instructions for maximizing the resolution of the data. In embodiments, maximizing the resolution of the data includes calculating a resolution score that accounts for the sum of resolution indices, the number of populations, the number of parameters and the number of cells. In some embodiments, a resolution score is calculated for each value of n (i.e., number of populations) such that there is a resolution score associated with each possible number of populations. i.e., to determine an optimal number and arrangement of population clusters that maximizes the resolution of the data. Where desired, the processor includes instructions for reducing the dimensionality of the data by subjecting the data to a dimensionality reduction algorithm that has been selected because it produces population clusters that possess a higher resolution score than other dimensionality reduction algorithms.

Aspects of the present disclosure also include non-transitory computer readable storage media. In some embodiments, computer readable storage media of interest include a computer program stored thereon, where the computer program when loaded on the computer includes instructions for classifying data according to one or more different parameters, detecting heterogeneity in the data by calculating a resolution index for any given number of adjacent first and second populations of data, calculating Hartigan's dip statistic for each population of data and generating an image composed of heatmaps or plots. Where desired, the computer readable storage medium of interest includes instructions for maximizing the resolution of the data. In embodiments, maximizing the resolution of the data includes calculating a resolution score that accounts for the sum of resolution indices, the number of populations, the number of parameters and the number of cells. In some embodiments, a resolution score is calculated for each value of n (i.e., number of populations) such that there is a resolution score associated with each possible number of populations. i.e., to determine an optimal number and arrangement of population clusters that maximizes the resolution of the data. Where desired, the computer readable storage medium includes instructions for reducing the dimensionality of the data by subjecting the data to a dimensionality reduction algorithm that has been selected because it produces population clusters that possess a higher resolution score than other dimensionality reduction algorithms.

In embodiments, the subject methods, systems and computer readable media are configured to analyze the data within a software or an analysis tool for analyzing flow cytometer data or nucleic acid sequence data, such as FlowJo® or SeqGeq® (Ashford, Oreg.). The instant methods, systems and computer readable media, or a portion thereof, can be implemented as software components of a software for analyzing data, such as FlowJo® or SeqGeq®. In these embodiments the subject methods, systems and computer readable media according to the instant disclosure may function as a software “plugin” for an existing software package, such as FlowJo® and SeqGeq®.

### BRIEF DESCRIPTION OF THE FIGURES

The invention may be best understood from the following detailed description when read in conjunction with the accompanying drawings. Included in the drawings are the following figures:

FIG. 1 depicts a sample calculation of a stain index that is conventionally used in data analysis.

FIG. 2 depicts measures of variability for populations of data presented on a two-dimensional scatterplot.

FIG. 3 depicts separation distances between populations of data presented on a two-dimensional scatterplot.

FIG. 4 presents a sample calculation of a resolution index.

FIG. 5 depicts a heatmap demonstrating the separation between populations of data as determined by the resolution index.

FIG. 6 depicts a heatmap demonstrating the modality of different populations of data as determined by Hartigan's dip statistic.

FIG. 7 depicts a flowchart that schematically demonstrates the calculation of the resolution index and its relationship to the resolution score.

FIG. 8 depicts a graph demonstrating how resolution scores vary as the number of population clusters changes.

FIG. 9 presents three different two-dimensional scatterplots, each demonstrating the result of a different dimensionality reduction algorithm.

FIG. 10 depicts a flow cytometer according to certain embodiments.

FIG. 11 depicts a functional block diagram for one example of a processor according to certain embodiments.

FIG. 12 depicts a block diagram of a computing system according to certain embodiments.

### DETAILED DESCRIPTION

Methods for detecting heterogeneity in data (e.g., flow cytometer data, nucleic acid sequence data) are provided. In some instances, methods include generating one or more population clusters based on the determined parameters of analytes (e.g., cells, particles, nucleic acids) in a biological sample. In embodiments, methods include calculating a resolution index by computing a ratio between measures of variability and separation distance for any given number of pairs of first and second populations of data. Where desired, methods also include maximizing the resolution between populations of data by computing a resolution score that accounts for the sum of resolution indices, the number of populations, the number of parameters and the number of cells. Systems and computer-readable media for determining heterogeneity between populations of data and, where desired, maximizing resolution between populations of data, are also provided.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs, Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### Methods for Detecting Heterogeneity in Data

As reviewed above, methods for detecting heterogeneity in data (e.g., between two different populations of data) are provided. By “detecting heterogeneity”, it is meant determining if two populations of data are sufficiently distinct such that they can be considered separate population dusters. In some embodiments, detecting heterogeneity in data includes determining the relatedness, or lack thereof, between populations of data. In other embodiments, detecting heterogeneity in data includes assessing the quality of data clustering. In embodiments, the quality of data clustering is assessed by determining if, in general, the resolution between different populations of flow is too low (i.e., populations are “overclustered” such that they are not properly distinguished).

Flow Cytometer Data

In some embodiments, the data analyzed in the instant method is flow cytometer data having parameters of particles in the sample is generated from detected light. By “flow cytometer data” it is meant information regarding parameters of the particles in the flow cell that is collected by any number of detectors in a flow cytometer. In embodiments, the flow cytometer data is received from a forward scatter detector. A forward scatter detector may, in some instances, yield information regarding the overall size of a particle, In embodiments, the flow cytometer data is received from a side scatter detector. A side scatter detector may, in some instances, be configured to detect refracted and reflected light from the surfaces and internal structures of the particle, which tends to increase with increasing particle complexity of structure. In embodiments, the flow cytometer data is received from a fluorescent light detector. A fluorescent light detector may, in some instances, be configured to detect fluorescence emissions from fluorescent molecules, e.g., labeled specific binding members (such as labeled antibodies that specifically bind to markers of interest) associated with the particle in the flow cell, In certain embodiments, methods include detecting fluorescence from the sample with one or more fluorescence detectors, such as 2 or more, such as 3 or more, such as 4 or more, such as 5 or more, such as 6 or more, such as 7 or more, such as 8 or more, such as 9 or more, such as 10 or more, such as 15 or more and including 25 or more fluorescence detectors, In embodiments, each of the fluorescence detectors is configured to generate a fluorescence data signal. Fluorescence from the sample may be detected by each fluorescence detector, independently, over one or more of the wavelength ranges of 200 nm-1200 nm. In some instances, methods include detecting fluorescence from the sample over a range of wavelengths, such as from 200 nm to 1200 nm, such as from 300 nm to 1100 nm, such as from 400 nm to 1000 nm, such as from 500 nm to 900 nm and including from 600 nm to 800 nm. In other instances, methods include detecting fluorescence with each fluorescence detector at one or more specific wavelengths. For example, the fluorescence may be detected at one or more of 450 nm, 518 nm, 519 nm, 561 nm, 578 nm, 605 nm, 607 nm, 625 nm, 650 nm, 660 nm, 667 nm, 670 nm, 668 nm, 695 nm, 710 nm, 723 nm, 780 nm, 785 nm, 647 nm, 617 nm and any combinations thereof, depending on the number of different fluorescence detectors in the subject light detection system. In certain embodiments, methods include detecting wavelengths of light which correspond to the fluorescence peak wavelength of certain fluorophores present in the sample. In embodiments, flow cytometer data is received from one or more light detectors (e.g., one or more detection channels), such as 2 or more, such as 3 or more, such as 4 or more, such as 5 or more, such as 6 or more and including 8 or more light detectors (e.g., 8 or more detection channels).

In practicing methods according to certain embodiments, a sample having particles is irradiated with a light source and light from the sample is detected to generate populations of related particles based at least in part on the measurements of the detected light. In some instances, the sample is a biological sample. The term “biological sample” is used in its conventional sense to refer to a whole organism, plant, fungi or a subset of animal tissues, cells or component parts which may in certain instances be found in blood, mucus, lymphatic fluid, synovial fluid, cerebrospinal fluid, saliva, bronchoalveolar lavage, amniotic fluid, amniotic cord blood, urine, vaginal fluid and semen. As such, a “biological sample” refers to both the native organism or a subset of its tissues as well as to a homogenate, lysate or extract prepared from the organism or a subset of its tissues, including but not limited to, for example, plasma, serum, spinal fluid, lymph fluid, sections of the skin, respiratory, gastrointestinal, cardiovascular, and genitourinary tracts, tears, saliva, milk, blood cells, tumors, organs. Biological samples may be any type of organismic tissue, including both healthy and diseased tissue (e.g., cancerous, malignant, necrotic, etc.). In certain embodiments, the biological sample is a liquid sample, such as blood or derivative thereof, e.g., plasma, tears, urine, semen, etc., where in some instances the sample is a blood sample, including whole blood, such as blood obtained from venipuncture or fingerstick (where the blood may or may not be combined with any reagents prior to assay, such as preservatives, anticoagulants, etc.).

In certain embodiments the source of the sample is a “mammal” or “mammalian”, where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In some instances, the subjects are humans. The methods may be applied to samples obtained from human subjects of both genders and at any stage of development (i.e., neonates, infant, juvenile, adolescent, adult), where in certain embodiments the human subject is a juvenile, adolescent or adult. While the present invention may be applied to samples from a human subject, it is to be understood that the methods may also be carried-out on samples from other animal subjects (that is, in “non-human subjects”) such as, but not limited to, birds, mice, rats, dogs, cats, livestock and horses.

In practicing the subject methods, a sample having particles (e.g., in a flow stream of a flow cytometer) is irradiated with light from a light source. In some embodiments, the light source is a broadband light source, emitting light having a broad range of wavelengths, such as for example, spanning 50 nm or more, such as 100 nm or more, such as 150 nm or more, such as 200 nm or more, such as 250 nm or more, such as 300 nm or more, such as 350 nm or more, such as 400 nm or more and including spanning 500 nm or more. For example, one suitable broadband light source emits light having wavelengths from 200 nm to 1500 nm. Another example of a suitable broadband light source includes a light source that emits light having wavelengths from 400 nm to 1000 nm. Where methods include irradiating with a broadband light source, broadband light source protocols of interest may include, but are not limited to, a halogen lamp, deuterium arc lamp, xenon arc lamp, stabilized fiber-coupled broadband light source, a broadband LED with continuous spectrum, superluminescent emitting diode, semiconductor light emitting diode, wide spectrum LED white light source, an mufti-LED integrated white light source, among other broadband light sources or any combination thereof.

In other embodiments, methods includes irradiating with a narrow band light source emitting a particular wavelength or a narrow range of wavelengths, such as for example with a light source which emits light in a narrow range of wavelengths like a range of 50 nm or less, such as 40 nm or less, such as 30 nm or less, such as 25 nm or less, such as 20 nm or less, such as 15 nm or less, such as 10 nm or less, such as 5 nm or less, such as 2 nm or less and including light sources which emit a specific wavelength of light (i.e., monochromatic light). Where methods include irradiating with a narrow band light source, narrow band light source protocols of interest may include, but are not limited to, a narrow wavelength LED, laser diode or a broadband light source coupled to one or more optical bandpass filters, diffraction gratings, monochromators or any combination thereof.

Nucleic Acid Sequence Data

In other embodiments, the data analyzed in the instant method is nucleic acid sequence data. By “nucleic acid sequence data” it is meant information regarding the sequence of one or more nucleic acid samples contained within a biological sample. As described in detail above in the Introduction section, nucleic acid sequencing methods include, e.g., “next-generation” sequencing methods, including those based on successive cycles of incorporation of fluorescently labeled nucleic acid analogues.

The nucleic acid sample may be any nucleic acid sample that includes, or is suspected of including, one or more nucleic acids of interest, e.g., one or more nucleic acids for which amplification of the one or more nucleic acids is desirable. Amplification of the one or more nucleic acids may be desirable for a variety of reasons, including but not limited to, sequencing the amplification products (or “amplicons”) of the one or more nucleic acids of interest. Sequencing the amplification products enables one to determine the nucleotide sequence(s) of the one or more nucleic acids of interest and, optionally, to quantify the amount of the one or more nucleic acids of interest present in the nucleic acid sample.

The nucleic acid sample may be one or more cells, or a nucleic acid sample isolated from one or more cells. For example, the nucleic acid sample may be a nucleic acid sample isolated from a single cell, a plurality of cells (e.g., cultured cells), a tissue, an organ, or an organism (e.g., bacteria, yeast, or the like). In certain aspects, the nucleic acid sample is isolated from a cell(s), tissue, organ, and/or the like of a mammal (e.g., a human, a rodent (e.g., a mouse), or any other mammal of interest). In other aspects, the nucleic acid sample is isolated from a source other than a mammal, such as bacteria, yeast, insects (e.g., drosophila), amphibians (e.g., frogs (e.g., Xenopus)), viruses, plants, or any other non-mammalian nucleic acid sample source.

According to certain embodiments, the nucleic acid sample is isolated from a biological sample, such as a biological fluid or a biological tissue. Examples of biological fluids include urine, blood, plasma, serum, saliva, semen, stool, sputum, cerebral spinal fluid, tears, mucus, sperm, amniotic fluid or the like. Biological tissues are aggregate of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cells.

In certain aspects, the nucleic acid sample is isolated from a microorganism. Microorganisms of interest include, e.g., bacteria, fungi, yeasts, protozoans, viruses (including both non-enveloped and enveloped viruses), bacterial endospores (for example, Bacillus (including Bacillus anthracis, Bacillus cereus, and Bacillus subtilis) and Clostridium (including Clostridium botulinum Clostridium difficile, and Clostridium perfringens)), and combinations thereof. Genera of microorganisms of interest include, but are not limited to, Listeria, Escherichia, Salmonella, Campylobacter, Clostridium, Helicobacter, Mycobacterium, Staphylococcus, Shigella, Enterococcus, Neisseria, Shigella, Streptococcus, Vibrio, Yersinia, Bordetella, Borrelia, Pseudomonas, Saccharomyces, Candida, and the like, and combinations thereof. Specific microorganism strains of interest include, but are not limited to, Escherichia coil, Yersinia enterocolitica, Yersinia pseudotuberculosis, Vibrio cholerae, Vibrio parahaemolyticus, Vibrio vulnificus, Listeria monocytogenes, Staphylococcus aureus, Salmonella enterica, Saccharomyces cerevisiae, Candida albicans, Staphylococcal enterotoxin ssp, Bacillus cereus, Bacillus anthraces, Bacillus atrophaeus, Bacillus subtilis, Clostridium perfringens, Clostridium botulinum, Clostridium difficile, Enterobacter sakazakii, Pseudomonas aeruginosa, and the like, and combinations thereof (preferably, Staphylococcus aureus, Salmonella enterica, Saccharomyces cerevisiae, Bacillus atrophaeus, Bacillus subtilis, Escherichia coli, human-infecting non-enveloped enteric viruses for which Escherichia coli bacteriophage is a surrogate, and combinations thereof).

According to certain embodiments, the nucleic acid sample is a tumor nucleic acid sample (that is, a nucleic acid sample isolated from a tumor). “Tumor”, as used herein, refers to all neoplastic cell growth and proliferation, whether malignant or benign, and all pre-cancerous and cancerous cells and tissues. The terms “cancer” and “cancerous” refer to or describe the physiological condition in mammals that is typically characterized by unregulated cell growth/proliferation. Examples of cancer include but are not limited to, carcinoma, lymphoma, blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma, various types of head and neck cancer, and the like.

According to certain embodiments, the nucleic acid sample is a deoxyribonucleic acid (DNA) sample. DNA samples of interest include, but are not limited to, genomic DNA samples, mitochondrial DNA samples, complementary DNA (cDNA, synthesized from any RNA or DNA of interest) samples, recombinant DNA samples (e.g., plasmid DNA samples), and any other DNA samples of interest.

In certain aspects, the nucleic acid sample is a ribonucleic acid (RNA) sample. RNA samples of interest include, but are not limited to, messenger RNA (mRNA) samples, small/short interfering RNA (siRNA) samples, microRNA (miRNA), and any other RNA samples of interest.

Approaches, reagents and kits for isolating DNA and RNA from sources of interest are known in the art and commercially available. For example, kits for isolating DNA from a source of interest include the DNeasy®, RNeasy®, QIAamp®, and QlAquick® nucleic acid isolation/purification kits by Qiagen, Inc. (Germantown, Md.); the DNAzol®, ChargeSwitch®, Purelink®, and GeneCatcher® nucleic acid isolation/purification kits by Life Technologies. Inc. (Carlsbad, Calif.); the NucleoMag®, NucleoSpin®, and NucleoBond® nucleic acid isolation/purification kits by Clontech Laboratories, Inc. (Mountain View, Calif). In certain aspects, the nucleic acid is isolated from a fixed biological sample, e.g., formalin-fixed, paraffin-embedded (FFPE) tissue. Genomic DNA and RNA from FFPE tissue may be isolated using commercially available kits—such as the AllPrep® DNA/RNA FFPE kit by Qiagen, Inc. (Germantown, Md.), the RecoverAll® Total Nucleic Acid Isolation kit for FFPE by Life Technologies, Inc. (Carlsbad, Calif), and the NucleoSpin® FFPE kits by Clontech Laboratories, Inc. (Mountain View, Calif.).

In certain aspects, it may be desirable to sequence the amplification products (e.g., using a Sanger sequencing system, a next generation sequencing (NGS) system, or the like), where the addition of one or more sequencing adapters to the amplification products is useful or necessary for sequencing on a particular sequencing system of interest, Accordingly, in certain aspects, the methods further include adding a sequencing adapter to the amplified one or more nucleic acids of interest and the amplified one or more competitive internal standard nucleic acids. Such a step may be performed whether or not the amplified one or more nucleic acids of interest and the amplified one or more competitive internal standard nucleic acids already include one or more sequencing adapters (e.g., by virtue of the one or more amplification primers including one or more sequencing adapters as described above). Sequencing adapters that may be added to the amplified one or more nucleic acids of interest and the amplified one or more competitive internal standard nucleic acids include, e.g., one or more capture domains, one or more sequencing primer binding domains, one or more barcode domains, one or more barcode sequencing primer binding domains, one or more molecular identification domains, a complement of any such domains, or any combination thereof. Further details regarding sequencing adapters are described hereinabove.

According to certain embodiments, the methods include subjecting the amplified one or more nucleic acids of interest and the amplified one or more competitive internal standard nucleic acids to restriction enzyme digestion conditions in which either the one or more competitive internal standard nucleic acids or the amplified one or more nucleic acids of interest are cleaved by a restriction enzyme present in the digestion reaction. As described above, a mismatch in a competitive internal standard nucleic acid may create/provide a restriction enzyme recognition site in the competitive internal standard nucleic acid that is not present in the corresponding nucleic acid of the nucleic acid sample. Alternatively, a mismatch in a competitive internal standard nucleic acid may result in the absence of a restriction enzyme recognition site in the competitive internal standard nucleic acid that is present in the corresponding nucleic acid of interest of the nucleic acid sample. In this way, the mismatch finds use, e.g., in enabling one to distinguish the amplified one or more nucleic acids of interest and the amplified one or more competitive internal standard nucleic acids based on whether the restriction enzyme digests the amplified one or more nucleic acids of interest or the amplified one or more competitive internal standard nucleic acids.

In certain aspects, the methods include adding a sequencing adapter to the amplified one or more nucleic acids of interest and the amplified one or more competitive internal standard nucleic acids, and subjecting the amplified one or more nucleic acids of interest and the amplified one or more competitive internal standard nucleic acids to restriction enzyme digestion conditions, in any order as desired.

According to certain embodiments, the methods include sequencing the amplified one or more nucleic acids of interest and the amplified one or more competitive internal standard nucleic acids. Such amplification products may be sequenced directly (optionally after a purification step), or may be modified prior to being sequenced. Modifications prior to sequencing include, but are not limited to, the addition of one or more sequencing adapters as described above, subjecting the amplicons to restriction enzyme digestion conditions as described above, and/or any other useful modifications for sequencing the amplicons on a sequencing platform of interest.

The sequencing may be carried out on any suitable sequencing platform, including a Sanger sequencing platform, a next generation sequencing (NGS) platform (e.g., using a next generation sequencing protocol), or the like. NGS sequencing platforms of interest include, but are not limited to, a sequencing platform provided by IIlumina® (e.g., the HiSeq™, MiSeq™ and/or Genome Analyzer™ sequencing systems); Ion Torrent™ (e.g., the Ion PGM™ and/or Ion Proton™ sequencing systems); Pacific Biosciences (e.g., the PACBIO RS II sequencing system); Life Technologies™ (e.g., a SOLiD sequencing system); Roche (e.g., the 454 GS FLX+ and/or GS Junior sequencing systems); or any other sequencing platform of interest. Detailed protocols for preparing the amplicons for sequencing (e.g., by further amplification (e.g., solid-phase amplification), or the like), sequencing the amplicons, and analyzing the sequencing data are available from the manufacturer of the sequencing system of interest.

Data Analysis

After data (e.g., flow cytometer data, nucleic acid sequence data) is produced (e.g., by a flow cytometer or nucleic acid sequencing platform), embodiments of the method include analyzing the data. In some embodiments, methods include generating one or more population clusters based on the determined parameters of analytes (e.g., cells, particles, nucleic acids) in the sample. As used herein, a “population”, or “subpopulation” of analytes, such as cells, nucleic acids or other particles, generally refers to a group of analytes that possess properties (for example, optical, impedance, or temporal properties) with respect to one or more measured parameters such that measured parameter data form a cluster in the data space. In embodiments, data is comprised of signals from any given number of different parameters, such as, for instance 2 or more, 3 or more, 4 or more, 5 or more, 6 or more, 7 or more, 8 or more, 9 or more, 10 or more, and including 20 or more. Thus, populations are recognized as clusters in the data. Conversely, each data cluster generally is interpreted as corresponding to a population of a particular type of cell or analyte, although clusters that correspond to noise or background typically also are observed. A cluster may be defined in a subset of the dimensions, e.g., with respect to a subset of the measured parameters, which corresponds to populations that differ in only a subset of the measured parameters or features extracted from the measurements of the cell, particle or nucleic acid.

In embodiments, methods include receiving data, calculating parameters of each analyte, and clustering together analytes based on the calculated parameters. For example, where the data is flow cytometer data, an experiment may include particles labeled by several fluorophores or fluorescently labeled antibodies, and groups of particles may be defined by populations corresponding to one or more fluorescent measurements. In the example, a first group may be defined by a certain range of light scattering for a first fluorophore, and a second group may be defined by a certain range of light scattering for a second fluorophore. If the first and second fluorophores are represented on an x and y axis, respectively, two different color-coded populations might appear to define each group of particles, if the information was to be graphically displayed. Any number of analytes may be assigned to a cluster, including 5 or more analytes, such as 10 or more analytes, such as 50 or more analytes, such as 100 or more analytes, such as 500 analytes and including 1000 analytes. In certain embodiments, the method groups together in a cluster rare events (e.g., rare cells in a sample, such as cancer cells) detected in the sample. In these embodiments, the analyte clusters generated may include 10 or fewer assigned analytes, such as 9 or fewer and including 5 or fewer assigned analytes.

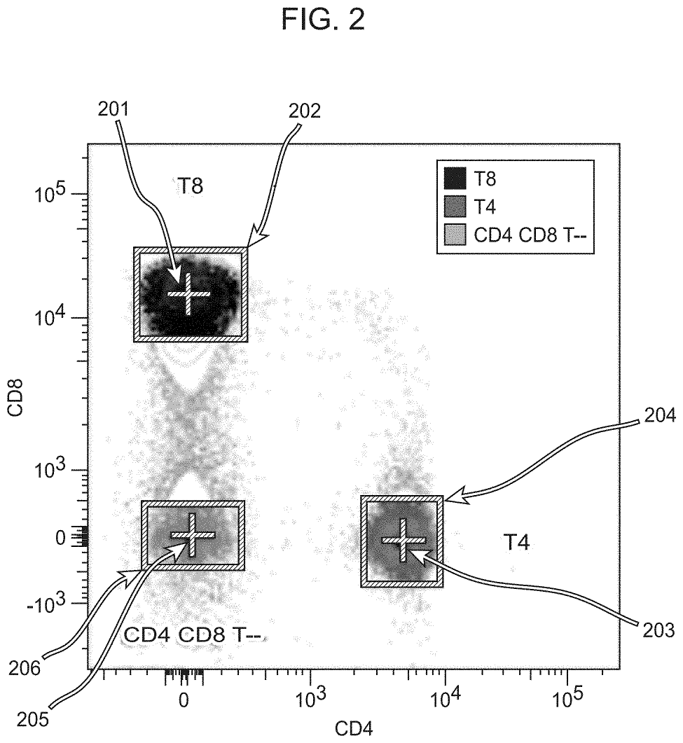

In embodiments, detecting heterogeneity in data includes obtaining measures of variability for first and second populations of data. By “measures of variability” it is meant mean and standard deviation. In some embodiments, the mean value is a mean centroid position of a given population of data in unidimensional or multidimensional space, and the standard deviation value is a measure of spread for a given population of data in unidimensional or multidimensional space. For example, FIG. 2 depicts obtaining measures of variability in two-dimensional space. The x-axis measures one parameter of flow cytometer data (i.e., presence of CD4) while the y-axis measures another parameter (i.e., presence of CD8). Three populations of flow cytometer data are depicted, The mean centroid positions 201, 203 and 205 are denoted by crosses in the centers of the first, second and third populations of flow cytometer data, respectively. Similarly, standard deviations 202, 204 and 206 are denoted by rectangles surrounding the first, second and third populations of data, respectively.

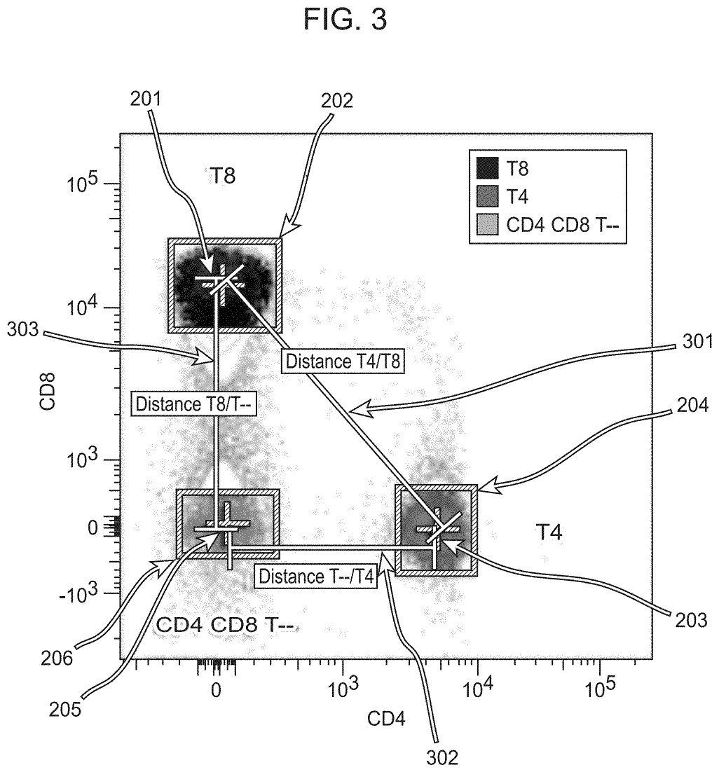

In some embodiments, detecting heterogeneity in data includes determining a separation distance between first and second populations of data. By “separation distance” it is meant the inter-population distance that separates pairs of populations. For example, FIG. 3 depicts the separation distance between the first, second and third populations of data depicted in FIG. 2. The separation distance between the first and second populations of flow cytometer data is defined by distance 301 drawn between mean centroid positions 201 and 203. The separation distance between the second and third populations of flow cytometer data is defined by distance 302 drawn between mean centroid positions 203 and 205. The separation distance between the third and first populations of flow cytometer data is defined by distance 303 drawn between mean centroid positions 205 and 201.

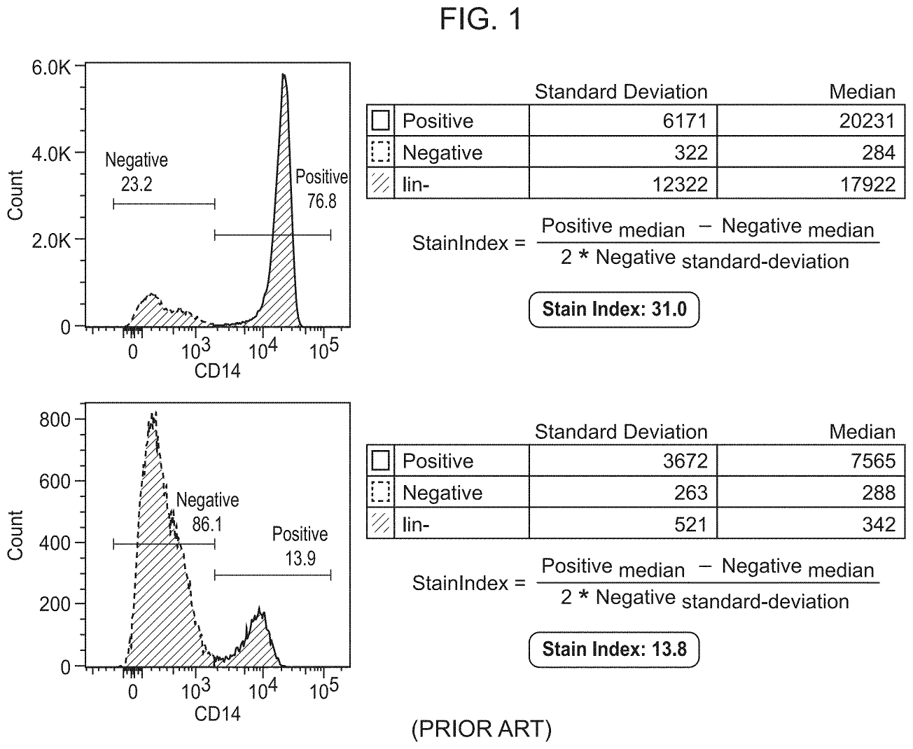

In some embodiments, the measures of variability and separation distance, e.g., as described herein, are used to calculate a resolution index, A resolution index as described herein is a quantification of the separation between the first and second populations of data. In embodiments, the resolution index provides a measure of heterogeneity for any given first and second populations of data. In embodiments, the resolution index provides an unbiased measure of separation between first and second populations of data by accounting for the intra-population variability of both populations (as opposed to measuring variability for only the negative population as shown in FIG. 1 with respect to the stain index). In embodiments where the first and second populations of data are positive and negative for a given parameter, respectively, the resolution index provides a measure of separation that accounts for the intra-population variability of both the positive and negative populations.

In some embodiments, the resolution index is determined by computing a ratio between the respective measures of variability for the first and second populations of data and the separation distance. In embodiments, the ratio is computed according to Equation A:

$$TaylorIndex = {TI}_{Clust 01 vsClust 02} = \frac{{\overline{X}}_{clust 01} - {\overline{X}}_{clust 02}}{{SD}_{clust 01} + {SD}_{clust 02}} \qquad ( A )$$

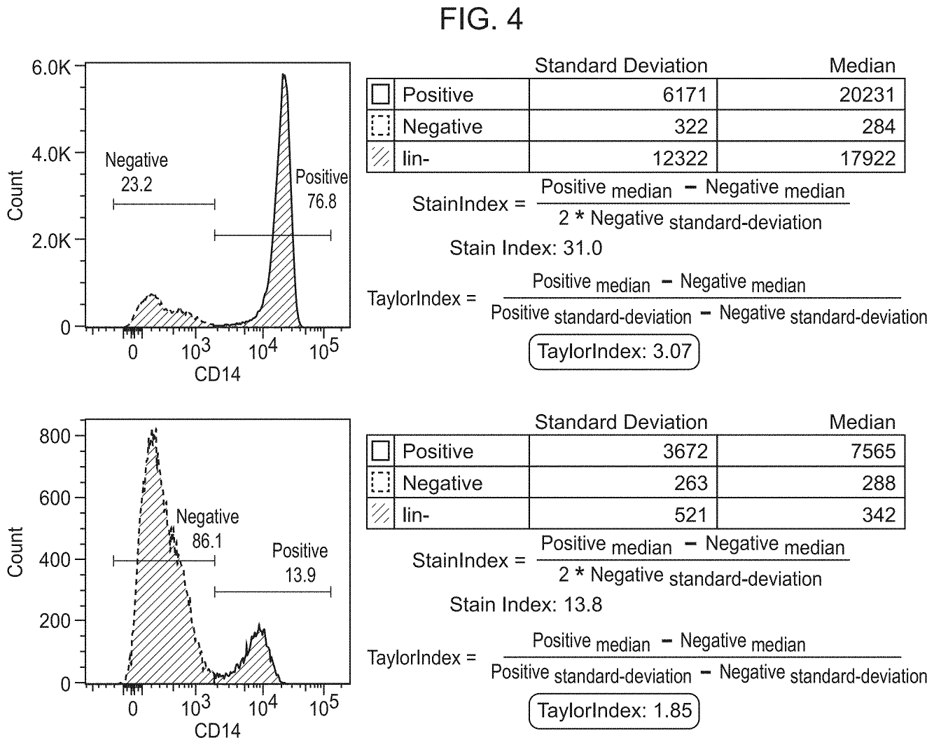

In Equation A, X̅_{clust01} is the mean centroid position of the first population of data, X̅_{clust02} is the mean centroid position of the second population of data, SD_{clust01} is the standard deviation of the first population of data, and SD_{clust02} is the standard deviation of the second population of data. In embodiments, a larger resolution index denotes a larger separation between two populations of data. Examples of resolution index calculations for positive and negative populations of data are presented in FIG. 4.

In certain embodiments, resolution indices are calculated for any given number of adjacent pairs of first and second populations of data, such as, for instance, 2 or more, 3 or more, 4 or more, 5 or more, 6 or more, 7 or more, 8 or more, 9 or more, 10 or more, 20 or more, 50 or more, and including 100 or more. In some embodiments, the number of adjacent pairs of data for which resolution indices are calculated is inputted by a user. In some embodiments, resolution indices are calculated for first and second populations of data that are defined by any given number of different parameters. For example, the populations of data described above in FIG. 2 and FIG. 3 are defined relative to their positions within two-dimensional space bounded by an x-axis and y-axis that each correspond to a different parameter (e.g., CD4 and CD8, respectively). However, some embodiments of the instant disclosure include calculating resolution indices for first and second populations of data that are defined within any given number of different dimensions, wherein each dimension is defined by a different parameter of data. In embodiments, resolution indices are calculated across 1 or more dimensions, 2 or more dimensions, 3 or more dimensions, 4 or more dimensions, 5 or more dimensions, 6 or more dimensions, 7 or more dimensions, 8 or more dimensions 9 or more dimensions, 10 or more dimensions, 15 or more dimensions, and including 20 or more dimensions.

In some embodiments, methods include calculating Hartigan's dip statistic for populations of data. The Hartigan's dip statistic as described herein is a known statistical test that checks for “saddle points” in data in order to determine if there is extra heterogeneity that might indicate the existence of deeper subpopulations of data. The Hartigan's dip statistic is described in detail in Jonathan B. Freeman & Rick Dale. Assessing bimodality to detect the presence of a dual cognitive process. Behav Res, 2012, the disclosure of which is herein incorporated by reference, In embodiments, computing a Hartigan's dip results in a modality score that provides a measure of how well data points associated with a given parameter are clustered together with a particular population of data. P-values associated with a modality score may range from 0 to 1. Lower P-values (e.g., <0.05) indicate significant multimodality (i.e., populations are less heterogeneous) while higher P-values provide evidence of unimodality (i.e., populations are more heterogeneous).

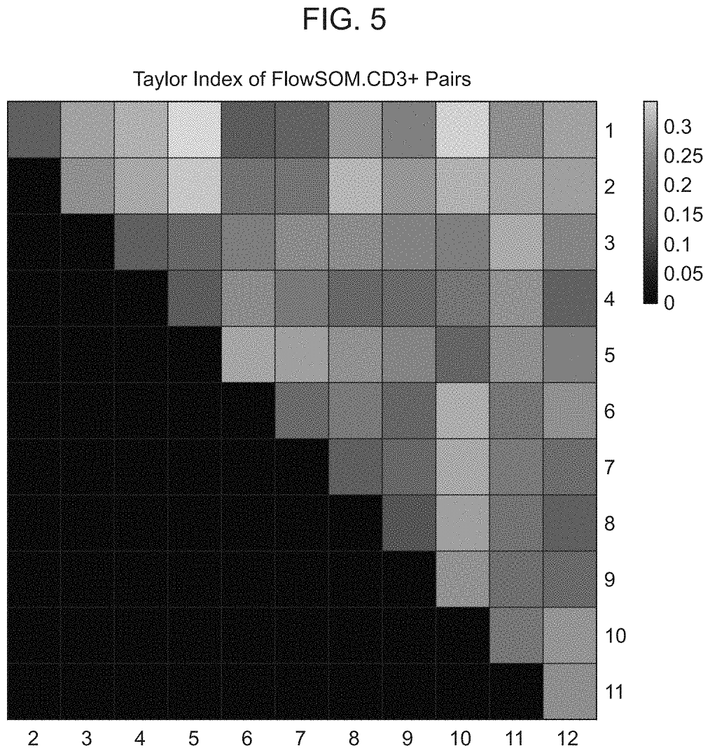

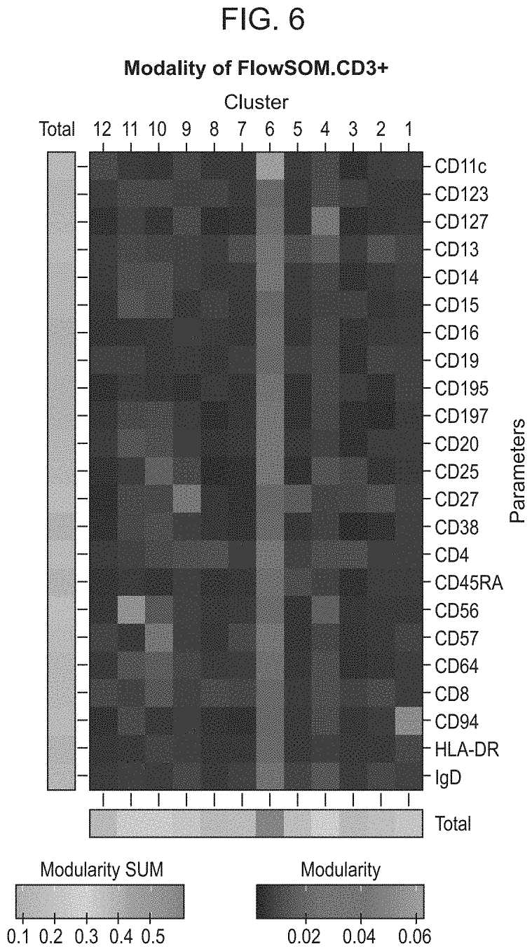

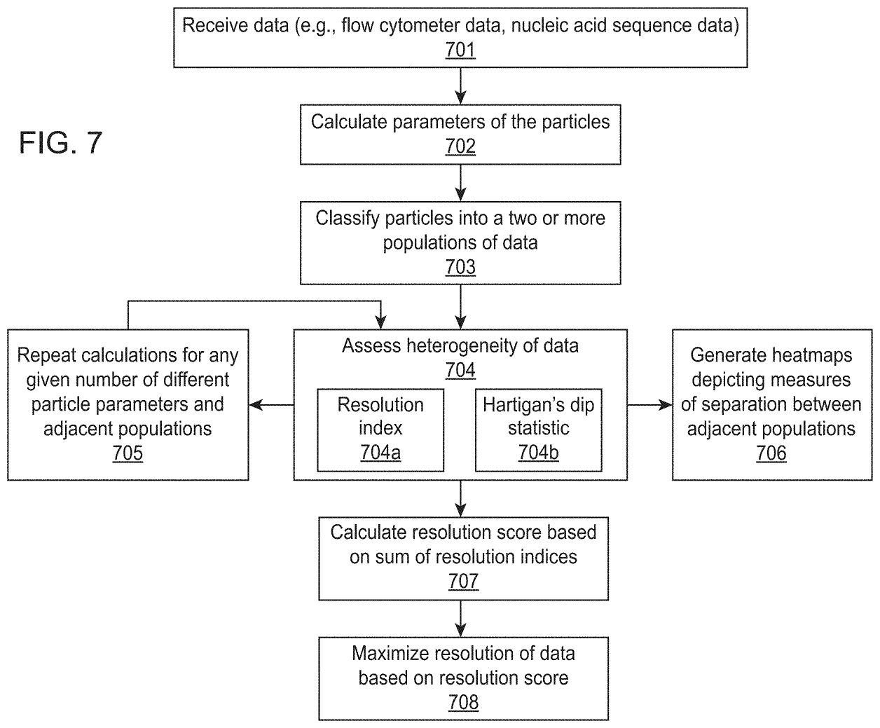

Aspects of the present disclosure also include generating an image that depicts, e.g., heterogeneity between any given number of first and second pairs of data. In some embodiments, generating an image includes generating a heatmap, i.e., a manner of visualizing data in which the magnitude of a phenomenon is related to a color. In embodiments, generating an image includes producing a heatmap composed of cells each containing a color, the intensity of which is related to the degree of heterogeneity between a particular pair of first and second populations of data as determined by the resolution index calculated for those two populations. In some embodiments, heatmaps as described herein also include a key that correlates a given color with a value of interest (e.g., resolution index). For example, FIG. 5 depicts a heatmap providing measures of heterogeneity between first and second populations of data. The horizontal and vertical axes each list a series of population clusters, and the cell in which the row and column for two population clusters overlaps contains a color, the intensity of which is related to the resolution index. In this example, a key is presented that associates a given resolution index with a given color. Embodiments of the invention also include producing a heatmap composed of cells each containing a color, the intensity of which is related to the degree of heterogeneity of a particular cluster of data as determined by the Hartigan's dip statistic (e.g., as discussed above). For example, FIG. 6 depicts a heatmap demonstrating the modularity of a given cluster (i.e., population) of data with respect to a given parameter. The horizontal axis lists a series of parameters, while the vertical axis lists a series of clusters. The cell in which a row (i.e., cluster) intersects with a column (i.e., parameter) provides a measure of modularity indicating how data points associated with a given parameter are clustered in a population of data. In this example, a key is presented that associates a given modality score with a given color. As discussed above, a lower modality score represents less heterogeneity in data, while a higher modality score represents more heterogeneity.

Where desired, methods also include maximizing resolution between populations of data. By “maximizing resolution” it is meant manipulating the data such that heterogenous populations of data (e.g., as determined above with the resolution index and Hartigan's dip statistic) are clearly separated into distinct population dusters. In certain embodiments, maximizing resolution of data includes calculating a resolution score. The “resolution score” as described herein provides a measure of separation between populations of data across any number of different parameters. In embodiments, the resolution score accounts for the number of parameters used for clustering, the number of cells analyzed, and the number of populations as well as their associated resolution indices (e.g., as calculated above for any given number of adjacent first and second populations of data). In some embodiments, the resolution score includes a summation of the resolution indices for each pair of first and second populations of data. For example, FIG. 7 presents a flowchart that schematically demonstrates the calculation of the resolution score. At step 701, data (e.g., flow cytometer data, nucleic acid sequence data) is received from an apparatus configured to analyze a biological sample (e.g., flow cytometer, nucleic acid sequencing platform).

Data is classified according to any given number of different parameters (step 702) and clustered into at least first and second populations of data at step 703 (e.g., as described above). At step 704, heterogeneity between the first and second populations of data are calculated by means of a resolution index (step 704a) and Hartigan's dip statistic (step 704b). Heterogeneity is similarly detected for any given number of other adjacent pairs of first and second populations of data (step 705). If desired, heatmaps may be generated in step 706 (e.g., as described above). When resolution indices are calculated for all desired pairs of first and second populations of data, the resolution indices are summed in a resolution score (step 707), which is then used to maximize the resolution of the data (step 708), In certain embodiments, the resolution score is calculated according to Equation B and Equation C:

$$\begin{matrix}TaylorFactor = TF = \frac{\sqrt{m}}{n \cdot p} 100 & ( B ) \\ TaylorScore = log [ {\sum }_{x = ClustPair}^{n choose 2} TI ( x ) ] \cdot {TF}^{AdjustmentFactor} & ( C )\end{matrix}$$

In Equation B and Equation C, TI is a resolution index, m is the number of cells, n is the number of populations, p is the number of parameters, AdjustmentFactor is a constant. In some embodiments, AdjustmentFactor is 0.7. In embodiments, maximizing the resolution of data includes calculating multiple resolution scores. In some embodiments, a resolution score is calculated for each value of n (i.e., number of populations) such that there is a resolution score associated with each possible number of populations. i.e., to determine an optimal number of population clusters with which data may be associated.

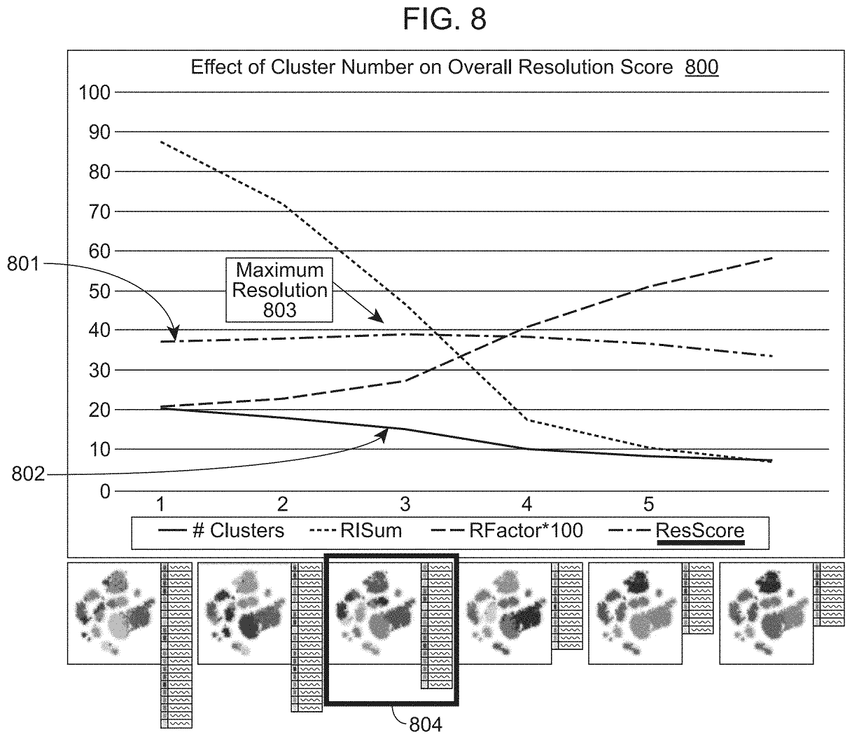

In embodiments, methods include altering the number of populations with which data points may be associated based on calculated resolution scores (described above). In other words, while data points themselves are not modulated, the manner in which they are associated with each other may be adjusted in order to optimize the resolution of the data. As such, data points may be associated with more or fewer population clusters depending on the resolution score calculated for each value of n (e.g., as described above). For example, FIG. 8 presents a graph 800 showing how resolution scores may vary as the number of population clusters changes. A curve 802 demonstrating change in number of population clusters is plotted with a curve 801 demonstrating change in the resolution score. As shown at point 803, there is a local maximum at which the resolution score is at its highest. Accordingly, the scatterplot 804 containing a number of population clusters that corresponds to the highest resolution score is selected as the arrangement of data in which resolution is at its maximum.

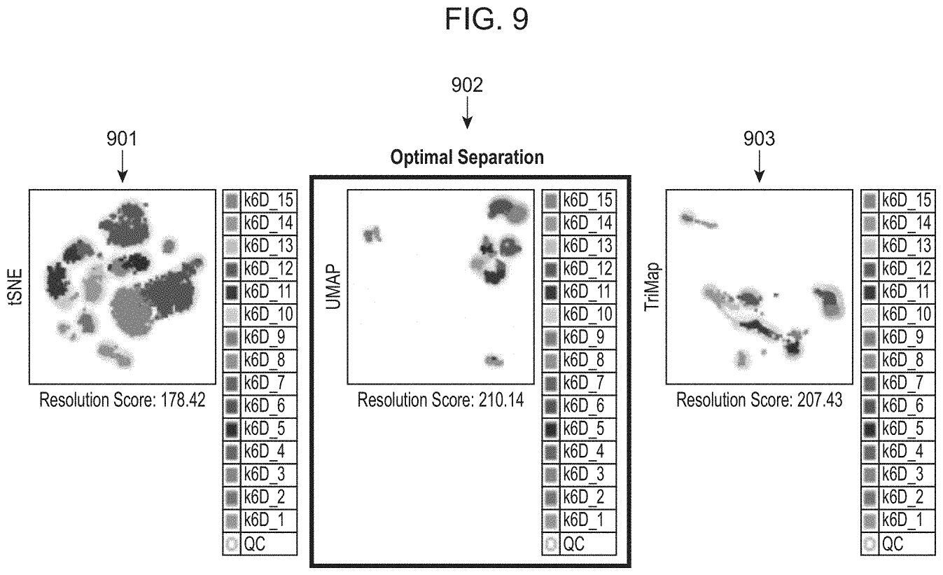

In certain embodiments, maximizing resolution in data includes dimensionality reduction. The term “dimensionality reduction” is used herein in its conventional sense to refer to manipulating a dataset such that the number of different variables under consideration are reduced. In some embodiments, dimensionality reduction includes performing a principal component analysis (PCA) that maps higher dimensional data onto lower dimensional space (e.g., two dimensions) such that the variance of the data in the lower dimensional space is maximized. Any suitable algorithm for dimensionality reduction may be used in the process of maximizing resolution. In some embodiments, dimensionality reduction is performed by a t-Distributed Stochastic Neighbor Embedding (t-SNE) algorithm. The t-SNE algorithm is described in Laurens van der Maaten & Geoffrey Hinton. Visualizing Data using t-SNE. Journal of Machine Learning Research, 2008; herein incorporated by reference. In some embodiments, dimensionality reduction is performed by a Uniform Manifold Approximation and Projection (UMAP) algorithm. The UMAP algorithm is described in Leland McInnes, John Healy & James Melville. UMAP: Uniform Manifold Approximation and Projection for Dimension Reduction. ARXIV, 2018; herein incorporated by reference. In some embodiments, dimensionality reduction is performed by a TriMap algorithm. The TriMap algorithm is described in Ehsan Amid & Manfred K. Warmuth TriMap: Large-scale Dimensionality Reduction Using Triplets. ARXIV, 2019; herein incorporated by reference. In some embodiments, resolution scores are calculated for each dimensionality reduction algorithm (e.g., t-SNE, UMAP and TriMap) such that the end product of each dimensionality reduction algorithm (e.g., a two-dimensional scatterplot) is evaluated for the manner in which datapoints contained therein are clustered into different populations. In other words, a given dimensionality reduction algorithm may produce population clusters that are more or less resolved (e.g., as determined by the resolution score) compared to another dimensionality reduction algorithm. In embodiments, methods include selecting and running a dimensionality reduction algorithm with the highest possible resolution score. For example, FIG. 9 presents 3 different two-dimensional scatterplots. Plot 901 is a result of the t-SNE dimensionality reduction algorithm, plot 902 is a result of the UMAP dimensionality reduction algorithm, and plot 903 is a result of the TriMap dimensionality reduction algorithm. As shown in FIG. 9, plot 902 (UMAP) provides a higher resolution score (i.e., 210.14) compared to either plot 901 or plot 903. As such, it is determined that the resolution of the population clustering for this particular dataset is highest when dimensionality reduction is performed by the UMAP dimensionality reduction algorithm.

### Systems for Detecting Heterogeneity in Data

Aspects of the present disclosure include systems for detecting heterogeneity in data and, where desired, maximizing the resolution between populations of data. In some embodiments, systems include an apparatus configured to produce data, and a processor configured to analyze the data.

Flow Cytometers

In certain embodiments, the apparatus configured to produce data is a flow cytometer. In some embodiments, the subject flow cytometers have a flow cell, and a laser configured to irradiate particles in the flow cell. In embodiments, the laser may be any convenient laser, such as a continuous wave laser. For example, the laser may be a diode laser, such as an ultraviolet diode laser, a visible diode laser and a near-infrared diode laser. In other embodiments, the laser may be a helium-neon (HeNe) laser. In some instances, the laser is a gas laser, such as a helium-neon laser, argon laser, krypton laser, xenon laser, nitrogen laser, CO₂ laser, CO laser, argon-fluorine (ArF) excimer laser, krypton-fluorine (KrF) excimer laser, xenon chlorine (XeCI) excimer laser or xenon-fluorine (XeF) excimer laser or a combination thereof. In other instances, the subject flow cytometers include a dye laser, such as a stilbene, coumarin or rhodamine laser. In yet other instances, lasers of interest include a metal-vapor laser, such as a helium-cadmium (HeCd) laser, helium-mercury (HeHg) laser, helium-selenium (HeSe) laser, helium-silver (HeAg) laser, strontium laser, neon-copper (NeCu) laser, copper laser or gold laser and combinations thereof. In still other instances, the subject flow cytometers include a solid-state laser, such as a ruby laser, an Nd:YAG laser, NdCrYAG laser, Er:YAG laser, Nd:YLF laser, Nd:YVO₄ laser, Nd:YCa₄O(BO₃)₃ laser, Nd:YCOB laser, titanium sapphire laser, thulim YAG laser, ytterbium YAG laser, ytterbium₂O₃ laser or cerium doped lasers and combinations thereof.

Aspects of the invention also include a forward scatter detector configured to detect forward scattered light. The number of forward scatter detectors in the subject flow cytometers may vary, as desired. For example, the subject flow cytometers may include 1 forward scatter detector or multiple forward scatter detectors, such as 2 or more, such as 3 or more, such as 4 or more, and including 5 or more. In certain embodiments, flow cytometers include 1 forward scatter detector. In other embodiments, flow cytometers include 2 forward scatter detectors.

Any convenient detector for detecting collected light may be used in the forward scatter detector described herein. Detectors of interest may include, but are not limited to, optical sensors or detectors, such as active-pixel sensors (APSs), avalanche photodiodes, image sensors, charge-coupled devices (CCDs), intensified charge-coupled devices (ICCDs), light emitting diodes, photon counters, bolometers, pyroelectric detectors, photoresistors, photovoltaic cells, photodiodes, photomultiplier tubes (PMTS), phototransistors, quantum dot photoconductors or photodiodes and combinations thereof, among other detectors. In certain embodiments, the collected light is measured with a charge-coupled device (CCD), semiconductor charge-coupled devices (CCD), active pixel sensors (APS), complementary metal-oxide semiconductor (CMOS) image sensors or N-type metal-oxide semiconductor (NMOS) image sensors. In certain embodiments, the detector is a photomultiplier tube, such as a photomultiplier tube having an active detecting surface area of each region that ranges from 0.01 cm² to 10 cm², such as from 0.05 cm² to 9 cm², such as from, such as from 0.1 cm² to 8 cm², such as from 0.5 cm² to 7 cm² and including from 1 cm² to 5 cm².

Where the subject flow cytometers include multiple forward scatter detectors, each detector may be the same, or the collection of detectors may be a combination of different types of detectors. For example, where the subject flow cytometers include two forward scatter detectors, in some embodiments the first forward scatter detector is a CCD-type device and the second forward scatter detector (or imaging sensor) is a CMOS-type device. In other embodiments, both the first and second forward scatter detectors are CCD-type devices. In yet other embodiments, both the first and second forward scatter detectors are CMOS-type devices. In still other embodiments, the first forward scatter detector is a CCD-type device and the second forward scatter detector is a photomultiplier tube (PMT). In still other embodiments, the first forward scatter detector is a CMOS-type device and the second forward scatter detector is a photomultiplier tube. In yet other embodiments, both the first and second forward scatter detectors are photomultiplier tubes.

In embodiments, the forward scatter detector is configured to measure light continuously or in discrete intervals. In some instances, detectors of interest are configured to take measurements of the collected light continuously. In other instances, detectors of interest are configured to take measurements in discrete intervals, such as measuring light every 0.001 millisecond, every 0.01 millisecond, every 0.1 millisecond, every 1 millisecond, every 10 milliseconds, every 100 milliseconds and including every 1000 milliseconds, or some other interval.

Embodiments of the invention also include a light dispersion/separator module positioned between the flow cell and the forward scatter detector. Light dispersion devices of interest include but are not limited to, colored glass, bandpass filters, interference filters, dichroic mirrors, diffraction gratings, monochromators and combinations thereof, among other wavelength separating devices, In some embodiments, a bandpass filter is positioned between the flow cell and the forward scatter detector, In other embodiments, more than one bandpass filter is positioned between the flow cell and the forward scatter detector, such as, for example, 2 or more, 3 or more, 4 or more, and including 5 or more, In embodiments, the bandpass filters have a minimum bandwidths ranging from 2 nm to 100 nm, such as from 3 nm to 95 nm, such as from 5 nm to 95 nm, such as from 10 nm to 90 nm, such as from 12 nm to 85 nm, such as from 15 nm to 80 nm and including bandpass filters having minimum bandwidths ranging from 20 nm to 50 nm. wavelengths and reflects light with other wavelengths to the forward scatter detector.

Certain embodiments of the invention include a side scatter detector configured to detect side scatter wavelengths of light (e.g._{;} light refracted and reflected from the surfaces and internal structures of the particle). In other embodiments, flow cytometers include multiple side scatter detectors, such as 2 or more, such as 3 or more, such as 4 or more, and including 5 or more.

Any convenient detector for detecting collected light may be used in the side scatter detector described herein. Detectors of interest may include, but are not limited to, optical sensors or detectors, such as active-pixel sensors (APSs), avalanche photodiodes, image sensors, charge-coupled devices (CCDs), intensified charge-coupled devices (ICCDs), light emitting diodes, photon counters, bolometers, pyroelectric detectors, photoresistors, photovoltaic cells, photodiodes, photomultiplier tubes (PMTS), phototransistors, quantum dot photoconductors or photodiodes and combinations thereof, among other detectors. In certain embodiments, the collected light is measured with a charge-coupled device (CCD), semiconductor charge-coupled devices (CCD), active pixel sensors (APS), complementary metal-oxide semiconductor (CMOS) image sensors or N-type metal-oxide semiconductor (NMOS) image sensors. In certain embodiments, the detector is a photomultiplier tube, such as a photomultiplier tube having an active detecting surface area of each region that ranges from 0.01 cm² to 10 cm²_{;} such as from 0.05 cm² to 9 cm², such as from, such as from 0.1 cm² to 8 cm², such as from 0.5 cm² to 7 cm² and including from 1 cm² to 5 cm².

Where the subject flow cytometers include multiple side scatter detectors, each side scatter detector may be the same, or the collection of side scatter detectors may be a combination of different types of detectors. For example, where the subject flow cytometers include two side scatter detectors, in some embodiments the first side scatter detector is a CCD-type device and the second side scatter detector (or imaging sensor) is a CMOS-type device. In other embodiments, both the first and second side scatter detectors are CCD-type devices. In yet other embodiments, both the first and second side scatter detectors are CMOS-type devices. In still other embodiments, the first side scatter detector is a CCD-type device and the second side scatter detector is a photomultiplier tube (PMT). In still other embodiments, the first side scatter detector is a CMOS-type device and the second side scatter detector is a photomultiplier tube. In yet other embodiments, both the first and second side scatter detectors are photomultiplier tubes.

Embodiments of the invention also include a light dispersion/separator module positioned between the flow cell and the side scatter detector. Light dispersion devices of interest include but are not limited to, colored glass, bandpass filters, interference filters, dichroic mirrors, diffraction gratings, monochromators and combinations thereof, among other wavelength separating devices.

In embodiments, the subject flow cytometers also include a fluorescent light detector configured to detect one or more fluorescent wavelengths of light. In other embodiments, flow cytometers include multiple fluorescent light detectors such as 2 or more, such as 3 or more, such as 4 or more, 5 or more and including 6 or more.

Any convenient detector for detecting collected light may be used in the fluorescent light detector described herein. Detectors of interest may include, but are not limited to, optical sensors or detectors, such as active-pixel sensors (APSs), avalanche photodiodes, image sensors, charge-coupled devices (CCDs), intensified charge-coupled devices (ICCDs), light emitting diodes, photon counters, bolometers, pyroelectric detectors, photoresistors, photovoltaic cells, photodiodes, photomultiplier tubes (PMTs), phototransistors, quantum dot photoconductors or photodiodes and combinations thereof, among other detectors. In certain embodiments, the collected light is measured with a charge-coupled device (CCD), semiconductor charge-coupled devices (CCD), active pixel sensors (APS), complementary metal-oxide semiconductor (CMOS) image sensors or N-type metal-oxide semiconductor (NMOS) image sensors. In certain embodiments, the detector is a photomultiplier tube, such as a photomultiplier tube having an active detecting surface area of each region that ranges from 0.01 cm² to 10 cm², such as from 0.05 cm² to 9 cm², such as from, such as from 0.1 cm² to 8 cm², such as from 0.5 cm² to 7 cm² and including from 1 cm² to 5 cm².

Where the subject flow cytometers include multiple fluorescent light detectors, each fluorescent light detector may be the same, or the collection of fluorescent light detectors may be a combination of different types of detectors. For example, where the subject flow cytometers include two fluorescent light detectors, in some embodiments the first fluorescent light detector is a CCD-type device and the second fluorescent light detector (or imaging sensor) is a CMOS-type device. In other embodiments, both the first and second fluorescent light detectors are CCD-type devices. In yet other embodiments, both the first and second fluorescent light detectors are CMOS-type devices. In still other embodiments, the first fluorescent light detector is a CCD-type device and the second fluorescent light detector is a photomultiplier tube (PMT). In still other embodiments, the first fluorescent light detector is a CMOS-type device and the second fluorescent light detector is a photomultiplier tube. In yet other embodiments, both the first and second fluorescent light detectors are photomultiplier tubes.

Embodiments of the invention also include a light dispersion/separator module positioned between the flow cell and the fluorescent light detector. Light dispersion devices of interest include but are not limited to, colored glass, bandpass filters, interference filters, dichroic mirrors, diffraction gratings, monochromators and combinations thereof, among other wavelength separating devices.

In embodiments of the present disclosure, fluorescent light detectors of interest are configured to measure collected light at one or more wavelengths, such as at 2 or more wavelengths, such as at 5 or more different wavelengths, such as at 10 or more different wavelengths, such as at 25 or more different wavelengths, such as at 50 or more different wavelengths, such as at 100 or more different wavelengths, such as at 200 or more different wavelengths, such as at 300 or more different wavelengths and including measuring light emitted by a sample in the flow stream at 400 or more different wavelengths. In some embodiments, 2 or more detectors in a flow cytometer as described herein are configured to measure the same or overlapping wavelengths of collected light.

In some embodiments, fluorescent light detectors of interest are configured to measure collected light over a range of wavelengths (e.g., 200nm-1000 nm). In certain embodiments, detectors of interest are configured to collect spectra of light over a range of wavelengths. For example, flow cytometers may include one or more detectors configured to collect spectra of light over one or more of the wavelength ranges of 200 nm-1000 nm. In yet other embodiments, detectors of interest are configured to measure light emitted by a sample in the flow stream at one or more specific wavelengths. For example, flow cytometers may include one or more detectors configured to measure light at one or more of 450 nm, 518 nm, 519 nm, 561 nm, 578 nm, 605 nm, 607 nm, 625 nm, 650 nm, 660 nm, 667 nm, 670 nm, 668 nm, 695 nm, 710 nm, 723 nm, 780 nm, 785 nm, 647 nm, 617 nm and any combinations thereof. In certain embodiments, one or more detectors may be configured to be paired with specific fluorophores, such as those used with the sample in a fluorescence assay.

Suitable flow cytometry systems may include, but are not limited to those described in Ormerod (ed.), Flow Cytometry: A Practical Approach, Oxford Univ. Press (1997); Jaroszeski et al. (eds.), Flow Cytometry Protocols, Methods in Molecular Biology No. 91, Humana Press (1997); Practical Flow Cytometry, 3rd ed., Wiley-Liss (1995); Virgo, et at (2012) Ann Clin Biochem. Jan; 49(pt 1):17-28; Linden, et. al., Semin Throm Hemost. 2004 Oct;30(5):502-11; Alison, et al, J Pathol, 2010 Dec; 222(4):335-344; and Herbig, et al. (2007) Crit Rev Ther Drug Carrier Cyst. 24(3):203-255; the disclosures of which are incorporated herein by reference. In certain instances, flow cytometry systems of interest include BD Biosciences FACSCanto™ II flow cytometer, BD Accuri™ flow cytometer, BD Biosciences FACSCelesta™ flow cytometer, BD Biosciences FACSLyric™ flow cytometer, BD Biosciences FACSVerse™ flow cytometer, BD Biosciences FACSymphony™ flow cytometer BD Biosciences LSRFortessa™ flow cytometer, BD Biosciences LSRFortess™ X-20 flow cytometer and BD Biosciences FACSCalibur™ cell sorter, a BD Biosciences FACSCount™ cell sorter, BD Biosciences FACSLyric™ cell sorter and BD Biosciences Via™ cell sorter BD Biosciences Influx™ cell sorter, BD Biosciences Jazz™ cell sorter, BD Biosciences Aria™ cell sorters and BD Biosciences FACSMelody™ cell sorter, or the like. In some embodiments, the subject particle sorting systems are flow cytometric systems, such those described in U.S. Pat. Nos. 9,952,076; 9 933,341; 9,726,527; 9,453,789; 9,200,334; 9,097,640; 9,095,494; 9,092,034; 8,975,595; 8,753,573; 8,233,146; 8,140,300; 7,544,326; 7,201,875; 7,129,505; 6,821,740; 6,813,017; 6,809,804; 6,372,506; 5,700,692; 5,643,796; 5,627,040; 5,620,842; 5,602,039; the disclosure of which are herein incorporated by reference in their entirety.

In some embodiments, particle sorting systems of interest are configured to sort particles with an enclosed particle sorting module, such as those described in U.S. Patent Publication No, 2017/0299493, filed on Mar. 28, 2017, the disclosure of which is incorporated herein by reference.

In certain embodiments, the subject particle systems are flow cytometric systems having an excitation module that uses radio-frequency multiplexed excitation to generate a plurality of frequency shifted beams of light, In these embodiments, the laser light generator may include a plurality of lasers and one or more acousto-optic components (e.g., an acoustooptic deflector, an acoustooptic frequency shifter) to generate a plurality of frequency shifted comb beams. In certain instances, the subject systems are flow cytometric systems having a laser excitation module as described in U.S. Pat. Nos. 9,423,353 and 9,784,661 and U.S. Patent Publication Nos. 2017/0133857 and 2017/0350803, the disclosures of which are herein incorporated by reference.

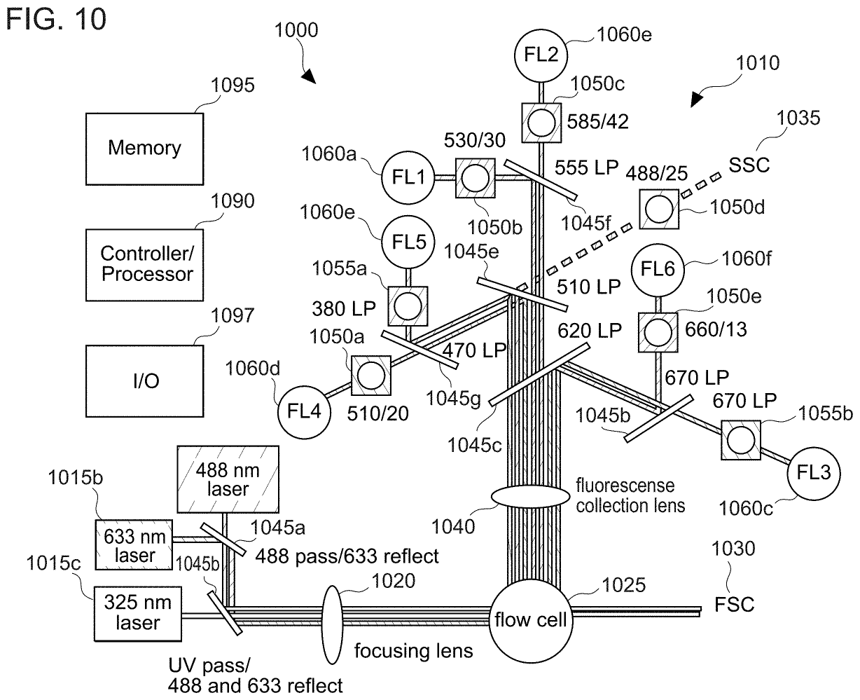

FIG. 10 shows a system 1000 for flow cytometry in accordance with an illustrative embodiment of the present invention. The system 1000 includes a flow cytometer 1010, a controller/processor 1090 and a memory 1095. The flow cytometer 1010 includes one or more excitation lasers 1015a-1015c, a focusing lens 1020, a flow chamber 1025, a forward scatter detector 1030, a side scatter detector 1035, a fluorescence collection lens 1040, one or more beam splitters 1045a-1045g, one or more bandpass filters 1050a-1050e, one or more longpass (“LP”) filters 1055a-1055b, and one or more fluorescent light detectors 1060a-1060f.

The excitation lasers 1015a-c emit light in the form of a laser beam. The wavelengths of the laser beams emitted from excitation lasers 1015a-1015c are 488 nm, 633 rim, and 325 nm, respectively, in the example system of FIG. 10. The laser beams are first directed through one or more of beam splitters 1045a and 1045b. Beam splitter 1045a transmits light at 488 nm and reflects light at 633 nm. Beam splitter 1045b transmits UV light (light with a wavelength in the range of 10 to 400 nm) and reflects light at 488 rim and 633 nm.

The laser beams are then directed to a focusing lens 1020, which focuses the beams onto the portion of a fluid stream where particles of a sample are located, within the flow chamber 1025. The flow chamber is part of a fluidics system which directs particles, typically one at a time, in a stream to the focused laser beam for interrogation. The flow chamber can comprise a flow cell in a benchtop cytometer or a nozzle tip in a stream-in-air cytometer.

The light from the laser beam(s) interacts with the particles in the sample by diffraction, refraction, reflection, scattering, and absorption with re-emission at various different wavelengths depending on the characteristics of the particle such as its size, internal structure, and the presence of one or more fluorescent molecules attached to or naturally present on or in the particle. The fluorescence emissions as well as the diffracted light, refracted light, reflected light, and scattered light may be routed to one or more of the forward scatter detector 1030, side scatter detector 1035, and the one or more fluorescent light detectors 1060a-1060f through one or more of the beam splitters 1045a-1045g, the bandpass filters 1050a-1050e, the longpass filters 1055a-1055b, and the fluorescence collection lens 1040.

The fluorescence collection lens 1040 collects light emitted from the particle-laser beam interaction and routes that light towards one or more beam splitters and filters. Bandpass filters, such as bandpass filters 1050a-1050e, allow a narrow range of wavelengths to pass through the filter. For example, bandpass filter 1050a is a 510/20 filter. The first number represents the center of a spectral band. The second number provides a range of the spectral band, Thus, a 510/20 filter extends 10 nm on each side of the center of the spectral band, or from 500 nm to 520 nm. Shortpass filters transmit wavelengths of light equal to or shorter than a specified wavelength. Longpass filters, such as longpass filters 1055a-1055b, transmit wavelengths of light equal to or longer than a specified wavelength of light, For example, longpass filter 1055a, which is a 670 nm longpass filter, transmits light equal to or longer than 670 nm. Filters are often selected to optimize the specificity of a detector for a particular fluorescent dye. The filters can be configured so that the spectral band of light transmitted to the detector is close to the emission peak of a fluorescent dye.

Beam splitters direct light of different wavelengths in different directions. Beam splitters can be characterized by filter properties such as shortpass and longpass. For example, beam splitter 10105g is a 620 SP beam splitter, meaning that the beam splitter 1045g transmits wavelengths of light that are 620 nm or shorter and reflects wavelengths of light that are longer than 620 nm in a different direction. In one embodiment, the beam splitters 1045a-1045g can comprise optical mirrors, such as dichroic mirrors.

The forward scatter detector 1030 is positioned off axis from the direct beam through the flow cell and is configured to detect diffracted light, the excitation light that travels through or around the particle in mostly a forward direction. The intensity of the light detected by the forward scatter detector is dependent on the overall size of the particle. The forward scatter detector can include a photodiode. The side scatter detector 1035 is configured to detect refracted and reflected light from the surfaces and internal structures of the particle, and tends to increase with increasing particle complexity of structure. The fluorescence emissions from fluorescent molecules associated with the particle can be detected by the one or more fluorescent light detectors 1060a-1060f. The side scatter detector 1035 and fluorescent light detectors can include photomultiplier tubes. The signals detected at the forward scatter detector 611, the side scatter detector 1035 and the fluorescent detectors can be converted to electronic signals (voltages) by the detectors. This data can provide information about the sample.

In operation, cytometer operation is controlled by a controller/processor 1090, and the measurement data from the detectors can be stored in the memory 1095 and processed by the controller/processor 1090. Although not shown explicitly, the controller/processor 1090 is coupled to the detectors to receive the output signals therefrom, and may also be coupled to electrical and electromechanical components of the flow cytometer 1000 to control the lasers, fluid flow parameters, and the like. Input/output (I/O) capabilities 1097 may be provided also in the system. The memory 1095, controller/processor 1090, and I/O 1097 may be entirely provided as an integral part of the flow cytometer 1010. In such an embodiment, a display may also form part of the I/O capabilities 1097 for presenting experimental data to users of the cytometer 1000. Alternatively, some or all of the memory 1095 and controller/processor 1090 and I/O capabilities may be part of one or more external devices such as a general purpose computer. In some embodiments, some or all of the memory 1095 and controller/processor 1090 can be in wireless or wired communication with the cytometer 1010. The controller/processor 1090 in conjunction with the memory 1095 and the I/O 1097 can be configured to perform various functions related to the preparation and analysis of a flow cytometer experiment.

The system illustrated in FIG. 10 includes six different detectors that detect fluorescent light in six different wavelength bands (which may be referred to herein as a “filter window” for a given detector) as defined by the configuration of filters and/or splitters in the beam path from the flow cell 1025 to each detector. Different fluorescent molecules used for a flow cytometer experiment will emit light in their own characteristic wavelength bands. The particular fluorescent labels used for an experiment and their associated fluorescent emission bands may be selected to generally coincide with the filter windows of the detectors. However, as more detectors are provided, and more labels are utilized, perfect correspondence between filter windows and fluorescent emission spectra is not possible. It is generally true that although the peak of the emission spectra of a particular fluorescent molecule may lie within the filter window of one particular detector, some of the emission spectra of that label will also overlap the filter windows of one or more other detectors. This may be referred to as spillover. The I/O 1097 can be configured to receive data regarding a flow cytometer experiment having a panel of fluorescent labels and a plurality of cell populations having a plurality of markers, each cell population having a subset of the plurality of markers. The I/O 1097 can also be configured to receive biological data assigning one or more markers to one or more cell populations, marker density data, emission spectrum data, data assigning labels to one or more markers, and cytometer configuration data. Flow cytometer experiment data, such as label spectral characteristics and flow cytometer configuration data can also be stored in the memory 1095. The controller/processor 1090 can be configured to evaluate one or more assignments of labels to markers.

One of skill in the art will recognize that a flow cytometer in accordance with an embodiment of the present invention is not limited to the flow cytometer depicted in FIG. 10, but can include any flow cytometer known in the art, For example, a flow cytometer may have any number of lasers, beam splitters, filters, and detectors at various wavelengths and in various different configurations.

Nucleic Acid Sequencing Platforms

In some embodiments, the apparatus is a nucleic acid sequencing platform. According to certain embodiments, the nucleic acid sequencing platforms find use in sequencing amplicons generated using the methods of the present disclosure.

In certain aspects, a sequencing system of the present disclosure includes a collection of nucleic acids. The collection of nucleic acids include amplicons corresponding to nucleic acids of interest present in a nucleic acid sample, and amplicons corresponding to a known amount of one or more competitive internal standard nucleic acids. The one or more competitive internal standard nucleic acids include a mismatch relative to one or more corresponding nucleic acids in the nucleic acid sample.

According to certain embodiments, the sequencing system includes amplicons generated from any of the one or more competitive internal standard nucleic acids and any of the nucleic acids of interest described above in the section relating to the methods of the present disclosure.

The amplicons may include a sequencing adapter provided during the amplification reaction that produced the amplicons (e.g., provided according to embodiments of the subject methods) and/or after the amplification reaction (e.g., provided according to embodiments of the subject methods). A subset of the amplicons (e.g., the amplified one or more competitive internal standard nucleic acids or the amplified one or more corresponding nucleic acids of interest) may be restriction enzyme digestion products, e.g., produced according to embodiments of the subject methods.

The sequencing system may be any sequencing system of interest, including a Sanger sequencing system, a next generation sequencing (NGS) system, or the like. In certain aspects the sequencing system is an NGS system. NGS systems of interest include, but are not limited to, a sequencing system provided by Illumina® (e.g., the HiSeq™, MiSeq™ and/or Genome Analyzer™ sequencing systems); Ion Torrent™ (e.g., the Ion PGM™ and/or Ion Proton™ sequencing systems); Pacific Biosciences (e.g., the PACBIO RS II sequencing system); Life Technologies™ (e.g., a SOLiD sequencing system); Roche (e.g., the 454 GS FLX+ and/or GS Junior sequencing systems), or any other suitable NGS systems.

The collection of nucleic acids may be present in a component of the sequencing system. By way of example, the collection of nucleic acids may be present in a sample preparation component of the sequencing system, e.g., a component of the sequencing system where nucleic acids of the collection are fragmented and/or sequencing adapters are added to the nucleic acids of the collection. Also, by way of example, the collection of nucleic acids may be present in a solid-phase amplification component of the sequencing system, where solid-phase amplification of the nucleic acids of the collection may occur. An example of such a solid-phase amplification component of a sequencing system is the flow cell of Iliumina-based sequencing systems, where cluster generation occurs. Another example of such a solid-phase amplification component of a sequencing system is the Ion OneTouch™ 2 component for producing templates suitable for sequencing on an Ion PGM™ system, Ion Proton™ system, or other NGS system provided by Ion Torrent™. The collection of nucleic acids may be present in any component of a sequencing system useful for utilizing the collection of nucleic acids to obtain the nucleic acid sequences thereof.

Processors

In certain embodiments, systems (e.g., flow cytometry systems, nucleic acid sequencing systems) are additionally include a processor having memory operably coupled to the processor wherein the memory includes instructions stored thereon, which when executed by the processor, cause the processor to detect heterogeneity in data (e.g., flow cytometry data, nucleic acid sequence data) and, where desired, maximize the resolution between populations of data.

In embodiments, after data (e.g., flow cytometer data, nucleic acid sequence data) is produced (e.g., by a flow cytometer or nucleic acid sequencing platform), the processor is configured to generate one or more population clusters based on the determined parameters of analytes (e.g., cells, particles, nucleic acids) in the sample. In these embodiments, the processor receives data, calculates parameters of each analyte, and clusters analytes together based on the calculated parameters. For example, where the data is flow cytometer data, an experiment may include particles labeled by several fluorophores or fluorescently labeled antibodies, and groups of particles may be defined by populations corresponding to one or more fluorescent measurements. In the example, a first group may be defined by a certain range of light scattering for a first fluorophore, and a second group may be defined by a certain range of light scattering for a second fluorophore. If the first and second fluorophores are represented on an x and y axis, respectively, two different color-coded populations might appear to define each group of particles, if the information was to be graphically displayed. Any number of analytes may be assigned to a cluster, including 5 or more analytes, such as 10 or more analytes, such as 50 or more analytes, such as 100 or more analytes, such as 500 analytes and including 1000 analytes. In certain embodiments, the method groups together in a cluster rare events (e.g., rare cells in a sample, such as cancer cells) detected in the sample. In these embodiments, the analyte clusters generated may include 10 or fewer assigned analytes, such as 9 or fewer and including 5 or fewer assigned analytes.

In embodiments, detecting heterogeneity in data includes obtaining measures of variability for first and second populations of data. By “measures of variability” it is meant mean and standard deviation. In some embodiments, the mean value is a mean centroid position of a given population of data in unidimensional or multidimensional space, and the standard deviation value is a measure of spread for a given population of data in unidimensional or multidimensional space. In some embodiments, detecting heterogeneity in data includes determining a separation distance between first and second populations of data By “separation distance” it is meant the inter-population distance that separates pairs of populations. In some embodiments, the measures of variability and separation distance, e.g., as described herein, are used to calculate a resolution index. A resolution index as described herein is a quantification of the separation between the first and second populations of data. In embodiments, the resolution index provides a measure of heterogeneity for any given first and second populations of data. In embodiments, the resolution index provides an unbiased measure of separation between first and second populations of data by accounting for the intra-population variability of both populations (as opposed to measuring variability for only the negative population as shown in FIG. 1 with respect to the stain index). In embodiments where the first and second populations of data are positive and negative for a given parameter, respectively, the resolution index provides a measure of separation that accounts for the intra-population variability of both the positive and negative populations.

In some embodiments, the resolution index is determined by computing a ratio between the respective measures of variability for the first and second populations of data and the separation distance. In embodiments, the ratio is computed according to Equation A;

$$TaylorIndex = {TI}_{Clust 01 vsClust 02} = \frac{{\overline{X}}_{clust 01} - {\overline{X}}_{clust 02}}{{SD}_{clust 01} + {SD}_{clust 02}} \qquad ( A )$$

In Equation A, X̅_{clust01} is the mean centroid position of the first population of data, X̅_{clust02} is the mean centroid position of the second population of data, SD_{clust01} is the standard deviation of the first population of data, and SD_{clust02} is the standard deviation of the second population of data. In embodiments, a larger resolution index denotes a larger separation between two populations of data.

In certain embodiments, resolution indices are calculated for any given number of adjacent pairs of first and second populations of data, such as, for instance, 2 or more, 3 or more, 4 or more, 5 or more, 6 or more, 7 or more, 8 or more, 9 or more, 10 or more, 20 or more, 50 or more, and including 100 or more. In some embodiments, the number of adjacent pairs of data for which resolution indices are calculated is inputted by a user. In some embodiments, the processor calculates resolution indices for first and second populations of data that are defined by any given number of different parameters. However, in some embodiments, the processor calculates resolution indices for first and second populations of data that are defined within any given number of different dimensions, wherein each dimension is defined by a different parameter of data. In embodiments, resolution indices are calculated across 1 or more dimensions, 2 or more dimensions, 3 or more dimensions, 4 or more dimensions, 5 or more dimensions, 6 or more dimensions, 7 or more dimensions, 8 or more dimensions 9 or more dimensions, 10 or more dimensions, 15 or more dimensions, and including 20 or more dimensions.

In some embodiments, the processor is configured to calculate Hartigan's dip statistic for populations of data. The Hartigan's dip statistic as described herein is a known statistical test that checks for “saddle points” in data in order to determine if there is extra heterogeneity that might indicate the existence of deeper subpopulations of data. In embodiments, computing a Hartigan's dip results in a modality score that provides a measure of how well data points associated with a given parameter are clustered together with a particular population of data. P-values associated with a modality score may range from 0 to 1. Lower P-values (e.g., <0.05) indicate significant multimodality (i.e., populations are less heterogeneous) while higher P-values provide evidence of unimodality (i.e., populations are more heterogeneous).

In some embodiments, the processor is configured to generate an image that depicts, e.g., heterogeneity between any given number of first and second pairs of data. In some embodiments, generating an image includes generating a heatmap, i.e., a manner of visualizing data in which the magnitude of a phenomenon is related to a color. In embodiments, generating an image includes producing a heatmap composed of cells each containing a color, the intensity of which is related to the degree of heterogeneity between a particular pair of first and second populations of data as determined by the resolution index calculated for those two populations. In some embodiments, heatmaps as described herein also include a key that correlates a given color with a value of interest (e.g., resolution index, Hartigan's dip statistic).

In some embodiments, the processor is configured to maximize resolution between populations of data. By “maximizing resolution” it is meant manipulating the data such that heterogenous populations of data (e.g., as determined above with the resolution index and Hartigan's dip statistic) are clearly separated into distinct population clusters. In certain embodiments, maximizing resolution of data includes calculating a resolution score. The “resolution score” as described herein provides a measure of separation between populations of data across any number of different parameters. In embodiments, the resolution score accounts for the number of parameters used for clustering, the number of cells analyzed, and the number of populations as well as their associated resolution indices (e.g., as calculated above for any given number of adjacent first and second populations of data). In some embodiments, the resolution score includes a summation of the resolution indices for each pair of first and second populations of data. In certain embodiments, the resolution score is calculated according to Equation B and Equation C:

$$\begin{matrix}TaylorFactor = TF = \frac{\sqrt{m}}{n \cdot p} 100 & ( B ) \\ TaylorScore = log [ {\sum }_{x = ClustPair}^{n choose 2} TI ( x ) ] \cdot {TF}^{AdjustmentFactor} & ( C )\end{matrix}$$

In Equation B and Equation C, TI is a resolution index, m is the number of cells, n is the number of populations, p is the number of parameters, AdjustmentFactor is a constant. In some embodiments, AdjustmentFactor is 0.7. In embodiments, maximizing the resolution of data includes calculating multiple resolution scores. In some embodiments, a resolution score is calculated for each value of n (i.e., number of populations) such that there is a resolution score associated with each possible number of populations. i.e., to determine an optimal number of population clusters with which data may be associated.

In embodiments, the processor is configured to alter the number of populations with which data points may be associated based on calculated resolution scores (described above). In other words, while data points themselves are not modulated, the manner in which they are associated with each other may be adjusted in order to optimize the resolution of the data. As such, data points may be associated with more or fewer population clusters depending on the resolution score calculated for each value of n (e.g., as described above).

In certain embodiments, maximizing resolution in data includes dimensionality reduction. The term “dimensionality reduction” is used herein in its conventional sense to refer to manipulating a dataset such that the number of different variables under consideration are reduced. In some embodiments, dimensionality reduction includes performing a principal component analysis (PCA) that maps higher dimensional data onto lower dimensional space (e.g., two dimensions) such that the variance of the data in the lower dimensional space is maximized. Any suitable algorithm for dimensionality reduction may be used in the process of maximizing resolution. In some embodiments, dimensionality reduction is performed by a t-Distributed Stochastic Neighbor Embedding (t-SNE) algorithm. In some embodiments, dimensionality reduction is performed by a Uniform Manifold Approximation and Projection (UMAP) algorithm. In some embodiments, dimensionality reduction is performed by a TriMap algorithm. In some embodiments, resolution scores are calculated for each dimensionality reduction algorithm (e.g., t-SNE, UMAP and TriMap) such that the end product of each dimensionality reduction algorithm (e.g., a two-dimensional scatterplot) is evaluated for the manner in which datapoints contained therein are clustered into different populations. In other words, a given dimensionality reduction algorithm may produce population clusters that are more or less resolved (e.g., as determined by the resolution score) compared to another dimensionality reduction algorithm. In embodiments, methods include selecting and running a dimensionality reduction algorithm with the highest possible resolution score.

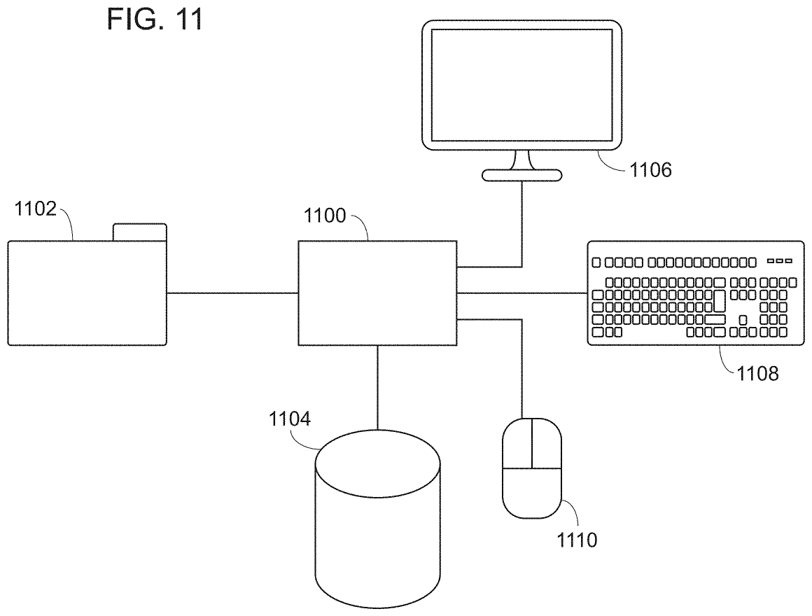

FIG. 11 shows a functional block diagram for one example of a processor 1100, for analyzing and displaying data. A processor 1100 can be configured to implement a variety of processes for controlling graphic display of biological events.

An apparatus 1102 can be configured to acquire data by analyzing a biological sample (e.g., as described above). For example, a flow cytometer can generate flow cytometer data. In addition, a nucleic acid sequencing system can be configured to generate nucleic acid sequence data. The apparatus can be configured to provide biological event data to the processor 1100. A data communication channel can be included between the apparatus 1102 and the processor 1100. The data can be provided to the processor 1100 via the data communication channel. In embodiments where the apparatus is a flow cytometer, data received from the apparatus 1102 includes flow cytometer data. In embodiments where the apparatus is a nucleic acid sequencing platform, data received from the apparatus 1102 includes nucleic acid sequencing data. The processor 1100 can be configured to provide a graphical display including heatmaps and plots (e.g., as described above) to display 1106. The processor 1100 can be further configured to render a gate around populations of data shown by the display device 1106, overlaid upon the plot, for example. In some embodiments, the gate can be a logical combination of one or more graphical regions of interest drawn upon a single parameter histogram or bivariate plot. In some embodiments, the display can be used to display analyte parameters or saturated detector data.

The processor 1100 can be further configured to display data on the display device 1106 within the gate differently from other events in the biological event data outside of the gate. For example, the processor 1100 can be configured to render the color of biological event data contained within the gate to be distinct from the color of biological event data outside of the gate. In this way, the processor 1100 may be configured to render different colors to represent each unique population of data. The display device 1106 can be implemented as a monitor, a tablet computer, a smartphone, or other electronic device configured to present graphical interfaces.

The processor 1100 can be configured to receive a gate selection signal identifying the gate from a first input device. For example, the first input device can be implemented as a mouse 1110. The mouse 1110 can initiate a gate selection signal to the processor 1100 identifying the population to be displayed on or manipulated via the display device 1106 (e.g., by clicking on or in the desired gate when the cursor is positioned there), In some implementations, the first device can be implemented as the keyboard 1108 or other means for providing an input signal to the processor 1100 such as a touchscreen, a stylus, an optical detector, or a voice recognition system. Some input devices can include multiple inputting functions. In such implementations, the inputting functions can each be considered an input device. For example, as shown in FIG. 11, the mouse 1110 can include a right mouse button and a left mouse button, each of which can generate a triggering event.

The triggering event can cause the processor 1100 to alter the manner in which the data is displayed, which portions of the data is actually displayed on the display device 1106, and/or provide input to further processing such as selection of a population of interest for analysis.

In some embodiments, the processor 1100 can be configured to detect when gate selection is initiated by the mouse 1110. The processor 1100 can be further configured to automatically modify plot visualization to facilitate the gating process. The modification can be based on the specific distribution of data received by the processor 1100.

The processor 1100 can be connected to a storage device 1104. The storage device 1104 can be configured to receive and store data from the processor 1100. The storage device 1104 can be further configured to allow retrieval of data, such as flow cytometric event data, by the processor 1100.

A display device 1106 can be configured to receive display data from the processor 1100. The display data can comprise plots of biological event data and gates outlining sections of the plots. The display device 1106 can be further configured to alter the information presented according to input received from the processor 1100 in conjunction with input from apparatus 1102, the storage device 1104, the keyboard 1108, and/or the mouse 1110.

In some implementations the processor 1100 can generate a user interface to receive example events for sorting. For example, the user interface can include a control for receiving example events or example images. The example events or images or an example gate can be provided prior to collection of event data for a sample, or based on an initial set of events for a portion of the sample.

### Computer-Controlled Systems

Aspects of the present disclosure further include computer-controlled systems. where the systems further include one or more computers for complete automation or partial automation. In some embodiments, systems include a computer having a computer readable storage medium with a computer program stored thereon, where the computer program when loaded on the computer includes instructions for classifying data according to one or more different parameters, detecting heterogeneity in the data by calculating a resolution index for any given number of adjacent first and second populations of data, calculating Hartigan's dip statistic for each population of data and generating an image composed of heatmaps or plots. Where desired, the computer program includes instructions for maximizing the resolution of the data. In embodiments. maximizing the resolution of the data includes calculating a resolution score that accounts for the sum of resolution indices, the number of populations, the number of parameters and the number of cells. In some embodiments, a resolution score is calculated for each value of n (i.e., number of populations) such that there is a resolution score associated with each possible number of populations. i.e., to determine an optimal number and arrangement of population clusters that maximizes the resolution of the data. Where desired, the computer program includes instructions for reducing the dimensionality of the data by subjecting the data to a dimensionality reduction algorithm that has been selected because it produces population clusters that possess a higher resolution score than other dimensionality reduction algorithms.

In embodiments, the system is configured to analyze the data within a software or an analysis tool for analyzing flow cytometer data or nucleic acid sequence data, such as FlowJo® or SeqGeq® (Ashland, Oreg.). FlowJo® is a software package developed by FlowJo LLC (a subsidiary of Becton Dickinson) for analyzing flow cytometer data. The software is configured to manage flow cytometer data and produce graphical reports thereon (https://www(dot)flowjo(dot)com/learn/flowjo-university/flowjo). SeqGeq® is a software package developed by FlowJo LLC (a subsidiary of Becton Dickinson) for analyzing gene expression data, especially from single-cell RNA sequencing. The software is configured to manage the gene expression data and produce graphical reports thereon (https://www(dot)flowjo(dot)com/learn/flowjo-university/seqgeq). The initial data can be analyzed within the data analysis software or tool (e.g., FlowJo®, SeqGeq®) by appropriate means, such as manual gating, cluster analysis, or other computational techniques. The instant systems, or a portion thereof, can be implemented as software components of a software for analyzing data, such as FlowJo® or SeqGeq®. In these embodiments, computer-controlled systems according to the instant disclosure may function as a software “plugin” for an existing software package, such as FlowJo® and SeqGeq®.

In embodiments, the system includes an input module, a processing module and an output module. The subject systems may include both hardware and software components, where the hardware components may take the form of one or more platforms, e.g., in the form of servers, such that the functional elements, i.e., those elements of the system that carry out specific tasks (such as managing input and output of information, processing information, etc.) of the system may be carried out by the execution of software applications on and across the one or more computer platforms represented of the system.

Systems may include a display and operator input device. Operator input devices may, for example, be a keyboard, mouse, or the like. The processing module includes a processor which has access to a memory having instructions stored thereon for performing the steps of the subject methods. The processing module may include an operating system, a graphical user interface (GUI) controller, a system memory, memory storage devices, and input-output controllers, cache memory, a data backup unit, and many other devices. The processor may be a commercially available processor or it may be one of other processors that are or will become available. The processor executes the operating system and the operating system interfaces with firmware and hardware in a well-known manner, and facilitates the processor in coordinating and executing the functions of various computer programs that may be written in a variety of programming languages, such as Java, Perl, C++, other high level or low level languages, as well as combinations thereof, as is known in the art. The operating system, typically in cooperation with the processor, coordinates and executes functions of the other components of the computer. The operating system also provides scheduling, input-output control, file and data management, memory management, and communication control and related services, all in accordance with known techniques. The processor may be any suitable analog or digital system. In some embodiments, processors include analog electronics which allows the user to manually align a light source with the flow stream based on the first and second light signals. In some embodiments, the processor includes analog electronics which provide feedback control, such as for example negative feedback control.

The system memory may be any of a variety of known or future memory storage devices. Examples include any commonly available random access memory (RAM), magnetic medium such as a resident hard disk or tape, an optical medium such as a read and write compact disc, flash memory devices, or other memory storage device. The memory storage device may be any of a variety of known or future devices, including a compact disk drive, a tape drive, a removable hard disk drive, or a diskette drive. Such types of memory storage devices typically read from, and/or write to, a program storage medium (not shown) such as, respectively, a compact disk, magnetic tape, removable hard disk, or floppy diskette. Any of these program storage media, or others now in use or that may later be developed, may be considered a computer program product. As will be appreciated, these program storage media typically store a computer software program and/or data Computer software programs, also called computer control logic, typically are stored in system memory and/or the program storage device used in conjunction with the memory storage device.

In some embodiments, a computer program product is described comprising a computer usable medium having control logic (computer software program, including program code) stored therein. The control logic, when executed by the processor the computer, causes the processor to perform functions described herein. In other embodiments, some functions are implemented primarily in hardware using, for example, a hardware state machine. Implementation of the hardware state machine so as to perform the functions described herein will be apparent to those skilled in the relevant arts.

Memory may be any suitable device in which the processor can store and retrieve data, such as magnetic, optical, or solid-state storage devices (including magnetic or optical disks or tape or RAM, or any other suitable device, either fixed or portable). The processor may include a general-purpose digital microprocessor suitably programmed from a computer readable medium carrying necessary program code. Programming can be provided remotely to processor through a communication channel, or previously saved in a computer program product such as memory or some other portable or fixed computer readable storage medium using any of those devices in connection with memory. For example, a magnetic or optical disk may carry the programming, and can be read by a disk writer/reader. Systems of the invention also include programming, e.g., in the form of computer program products, algorithms for use in practicing the methods as described above. Programming according to the present invention can be recorded on computer readable media, e.g., any medium that can be read and accessed directly by a computer, Such media include, but are not limited to: magnetic storage media, such as floppy discs, hard disc storage medium, and magnetic tape; optical storage media such as CD-ROM; electrical storage media such as RAM and ROM; portable flash drive; and hybrids of these categories such as magnetic/optical storage media.

The processor may also have access to a communication channel to communicate with a user at a remote location. By remote location is meant the user is not directly in contact with the system and relays input information to an input manager from an external device, such as a computer connected to a Wide Area Network (“WAN”), telephone network, satellite network, or any other suitable communication channel, including a mobile telephone (i.e., smartphone).

In some embodiments, systems according to the present disclosure may be configured to include a communication interface. In some embodiments, the communication interface includes a receiver and/or transmitter for communicating with a network and/or another device. The communication interface can be configured for wired or wireless communication, including, but not limited to, radio frequency (RF) communication (e.g., Radio-Frequency Identification (RFID), Zigbee communication protocols, WiFi, infrared, wireless Universal Serial Bus (USB), Ultra Wide Band (UWB), Bluetooth® communication protocols, and cellular communication, such as code division multiple access (CDMA) or Global System for Mobile communications (GSM).

In one embodiment, the communication interface is configured to include one or more communication ports, e.g., physical ports or interfaces such as a USB port, an RS-232 port, or any other suitable electrical connection port to allow data communication between the subject systems and other external devices such as a computer terminal (for example, at a physician's office or in hospital environment) that is configured for similar complementary data communication.

In one embodiment, the communication interface is configured for infrared communication, Bluetooth® communication, or any other suitable wireless communication protocol to enable the subject systems to communicate with other devices such as computer terminals and/or networks, communication enabled mobile telephones, personal digital assistants, or any other communication devices which the user may use in conjunction.

In one embodiment, the communication interface is configured to provide a connection for data transfer utilizing Internet Protocol (IP) through a cell phone network, Short Message Service (SMS), wireless connection to a personal computer (PC) on a Local Area Network (LAN) which is connected to the internet, or WiFi connection to the internet at a WiFi hotspot.

In one embodiment, the subject systems are configured to wirelessly communicate with a server device via the communication interface, e.g., using a common standard such as 802.11 or Bluetooth® RF protocol, or an IrDA infrared protocol. The server device may be another portable device, such as a smart phone, Personal Digital Assistant (PDA) or notebook computer; or a larger device such as a desktop computer, appliance, etc, In some embodiments, the server device has a display, such as a liquid crystal display (LCD), as well as an input device, such as buttons, a keyboard, mouse or touch-screen.

In some embodiments, the communication interface is configured to automatically or semi-automatically communicate data stored in the subject systems, e.g., in an optional data storage unit, with a network or server device using one or more of the communication protocols and/or mechanisms described above.

Output controllers may include controllers for any of a variety of known display devices for presenting information to a user, whether a human or a machine, whether local or remote. If one of the display devices provides visual information, this information typically may be logically and/or physically organized as an array of picture elements. A graphical user interface (GUI) controller may include any of a variety of known or future software programs for providing graphical input and output interfaces between the system and a user, and for processing user inputs. The functional elements of the computer may communicate with each other via system bus. Some of these communications may be accomplished in alternative embodiments using network or other types of remote communications. The output manager may also provide information generated by the processing module to a user at a remote location, e.g., over the Internet, phone or satellite network, in accordance with known techniques. The presentation of data by the output manager may be implemented in accordance with a variety of known techniques. As some examples, data may include SQL, HTML or XML documents, email or other files, or data in other forms. The data may include Internet URL addresses so that a user may retrieve additional SQL, HTML, XML, or other documents or data from remote sources. The one or more platforms present in the subject systems may be any type of known computer platform or a type to be developed in the future, although they typically will be of a class of computer commonly referred to as servers. However, they may also be a main-frame computer, a work station, or other computer type. They may be connected via any known or future type of cabling or other communication system including wireless systems, either networked or otherwise. They may be co-located or they may be physically separated. Various operating systems may be employed on any of the computer platforms, possibly depending on the type and/or make of computer platform chosen. Appropriate operating systems include Windows NT, Windows XP, Windows 7, Windows 8, iOS, Sun Solaris, Linux, OS/400, Compaq Tru64 Unix, SGI IRIX, Siemens Reliant Unix, and others.

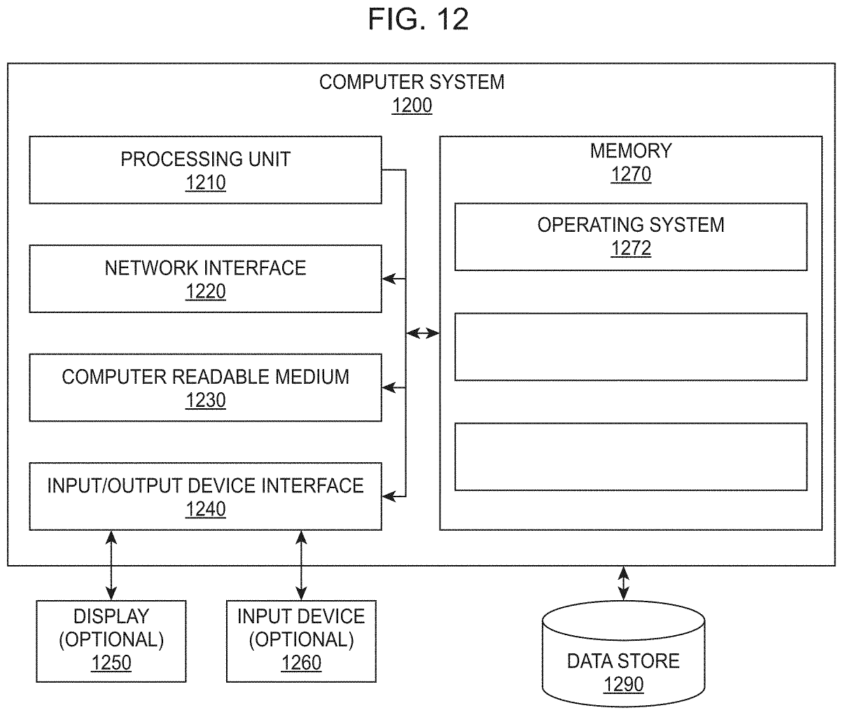

FIG. 12 depicts a general architecture of an example computing device 1200 according to certain embodiments. The general architecture of the computing device 1200 depicted in FIG. 12 includes an arrangement of computer hardware and software components. It is not necessary, however, that all of these generally conventional elements be shown in order to provide an enabling disclosure. As illustrated, the computing device 1200 includes a processing unit 1210, a network interface 1220, a computer readable medium drive 1230, an input/output device interface 1240, a display 1250, and an input device 1260, all of which may communicate with one another by way of a communication bus. The network interface 1220 may provide connectivity to one or more networks or computing systems. The processing unit 1210 may thus receive information and instructions from other computing systems or services via a network. The processing unit 1210 may also communicate to and from memory 1270 and further provide output information for an optional display 1250 via the input/output device interface 1240. For example, an analysis software (e.g., data analysis software or program such as FlowJo® and SeqGeq®) stored as executable instructions in the non-transitory memory of the analysis system can display the flow cytometry event data to a user, The input/output device interface 1240 may also accept input from the optional input device 1260, such as a keyboard, mouse, digital pen, microphone, touch screen, gesture recognition system, voice recognition system, gamepad, accelerometer, gyroscope, or other input device.

The memory 1270 may contain computer program instructions (grouped as modules or components in some embodiments) that the processing unit 1210 executes in order to implement one or more embodiments. The memory 1270 generally includes RAM, ROM and/or other persistent, auxiliary or non-transitory computer-readable media. The memory 1270 may store an operating system 1272 that provides computer program instructions for use by the processing unit 1210 in the general administration and operation of the computing device 1200. Data may be stored in data storage device 1290. The memory 1270 may further include computer program instructions and other information for implementing aspects of the present disclosure.

### Computer-Readable Storage Medium

Aspects of the present disclosure further include non-transitory computer readable storage mediums having instructions for practicing the subject methods. Computer readable storage media may be employed on one or more computers for complete automation or partial automation of a system for practicing methods described herein. In some embodiments, instructions in accordance with the method described herein can be coded onto a computer-readable medium in the form of “programming”, where the term “computer readable medium” as used herein refers to any non-transitory storage medium that participates in providing instructions and data to a computer for execution and processing. Examples of suitable non-transitory storage media include a floppy disk, hard disk, optical disk, magneto-optical disk, CD-ROM, CD-R, magnetic tape, non-volatile memory card, ROM, DVD-ROM, Blue-ray disk, solid state disk, and network attached storage (NAS), whether or not such devices are internal or external to the computer. In some instances, instructions may be provided on an integrated circuit device. Integrated circuit devices of interest may include, in certain instances, a reconfigurable field programmable gate array (FPGA), an application specific integrated circuit (ASIC) or a complex programmable logic device (CPLD). A file containing information can be “stored” on computer readable medium, where “storing” means recording information such that it is accessible and retrievable at a later date by a computer. The computer-implemented method described herein can be executed using programming that can be written in one or more of any number of computer programming languages. Such languages include, for example, Java (Sun Microsystems, Inc., Santa Clara, Calif.), Visual Basic (Microsoft Corp., Redmond, Wash.), and C++ (AT&T Corp., Bedminster, N.J.), as well as any many others.

In some embodiments, computer readable storage media of interest include a computer program stored thereon, where the computer program when loaded on the computer includes instructions for classifying data according to one or more different parameters, detecting heterogeneity in the data by calculating a resolution index for any given number of adjacent first and second populations of data, calculating Hartigan's dip statistic for each population of data and generating an image composed of heatmaps or plots. Where desired, the computer readable storage medium of interest includes instructions for maximizing the resolution of the data. In embodiments, maximizing the resolution of the data includes calculating a resolution score that accounts for the sum of resolution indices, the number of populations, the number of parameters and the number of cells. In some embodiments, a resolution score is calculated for each value of n (i.e., number of populations) such that there is a resolution score associated with each possible number of populations, i.e., to determine an optimal number and arrangement of population clusters that maximizes the resolution of the data. Where desired, the instant computer readable storage medium includes instructions for reducing the dimensionality of the data by subjecting the data to a dimensionality reduction algorithm that has been selected because it produces population clusters that possess a higher resolution score than other dimensionality reduction algorithms.

In embodiments, the system is configured to analyze the data within a software or an analysis tool for analyzing flow cytometer data or nucleic acid sequence data, such as FlowJo® or SeqGeq®. The initial data can be analyzed within the data analysis software or tool (e.g., FlowJo®, SeqGeq®) by appropriate means, such as manual gating, cluster analysis, or other computational techniques. The instant systems, or a portion thereof, can be implemented as software components of a software for analyzing data, such as FlowJo® or SeqGeq®. In these embodiments, computer-controlled systems according to the instant disclosure may function as a software “plugin” for an existing software package, such as FlowJo® and SeqGeq®.

The computer readable storage medium may be employed on more or more computer systems having a display and operator input device. Operator input devices may, for example, be a keyboard, mouse, or the like. The processing module includes a processor which has access to a memory having instructions stored thereon for performing the steps of the subject methods. The processing module may include an operating system, a graphical user interface (GUI) controller, a system memory, memory storage devices, and input-output controllers, cache memory, a data backup unit, and many other devices. The processor may be a commercially available processor, or it may be one of other processors that are or will become available. The processor executes the operating system and the operating system interfaces with firmware and hardware in a well-known manner, and facilitates the processor in coordinating and executing the functions of various computer programs that may be written in a variety of programming languages, such as Java, Perl, Python, C++, other high level or low level languages, as well as combinations thereof, as is known in the art. The operating system also provides scheduling, input-output control, file and data management, memory management, and communication control and related services, all in accordance with known techniques.

### Utility

The subject devices, methods and computer systems find use in a variety of applications where it is desirable to increase resolution and accuracy in the determination of parameters for analytes (e.g., cells, particles, nucleic acids) in a biological sample. For example, the present disclosure finds use in detecting heterogeneity between first and second populations of data in order to, e.g., determine if those populations are to be considered two, separate, populations. The subject devices, methods and computer systems also find use in identifying subpopulations of data (e.g., by comparing resolution index data to Hartigan's dip statistic data) that would otherwise not be identifiable. Where it is desired to maximize the resolution between populations of data, the subject devices, methods and computer systems find use in determining an arrangement of data (i.e., number of populations, dimensionality reduction) that maximizes the resolution between populations of data that are considered separable. In some embodiments, the subject methods and systems provide fully automated protocols so that adjustments to data require little, if any, human input.

The present disclosure can be employed to characterize many types of analytes, in particular, analytes relevant to medical diagnosis or protocols for caring for a patient, including but not limited to: proteins (including both free proteins and proteins and proteins bound to the surface of a structure, such as a cell), nucleic acids, viral particles, and the like. Further, samples can be from in vitro or in vivo sources, and samples can be diagnostic samples.

### Kits

Aspects of the present disclosure further include kits, where kits include storage media such as a floppy disk, hard disk, optical disk, magneto-optical disk, CD-ROM, CD-R, magnetic tape, non-volatile memory card, ROM, DVD-ROM, Blue-ray disk, solid state disk, and network attached storage (NAS). Any of these program storage media, or others now in use or that may later be developed, may be included in the subject kits. In embodiments, the program storage media include instructions for detecting heterogeneity between populations of first and second population of data, as well as maximizing the resolution between populations of data (e.g., as described above). In embodiments, the instructions contained on computer readable media provided in the subject kits, or a portion thereof, can be implemented as software components of a software for analyzing data, such as FlowJo® or SeqGeq®. In these embodiments, computer-controlled systems according to the instant disclosure may function as a software “plugin” for an existing software package, such as FlowJo®, and SeqGeq®.

In addition to the above components, the subject kits may further include (in some embodiments) instructions, e.g., for installing the plugin to the existing software package such as FlowJo® and SeqGeq®. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), portable flash drive, and the like, on which the information has been recorded. Yet another form of these instructions that may be present is a website address which may be used via the internet to access the information at a removed site.

Although the foregoing invention has been described in some detail by way of illustration and example for purposes of clarity of understanding, it is readily apparent to those of ordinary skill in the art in light of the teachings of this invention that some changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

1. A method of detecting heterogeneity in data, the method comprising:
obtaining measures of variability for first and second populations of data, respectively;
determining a separation distance for the first and second populations of data from the obtained measures of variability; and
calculating a resolution index of the first and second populations of data by comparing their respective measures of variability with the separation distance.

2. The method according to claim 1, wherein the data is flow cytometer data.

3. The method according to claim 1, wherein the data is nucleic acid sequencing data.

4. The method according to claim 1, wherein the resolution index is a quantification of the separation between the first and second populations of data.

5. The method according to claim 1, wherein the first population comprises data that is positive for a given parameter, and the second population comprises data that is negative for a given parameter.

6. The method according to claim 1, wherein obtaining measures of variability comprises calculating the mean centroid position and standard deviation for the first and second populations of data, respectively.

7. The method according to claim 1, wherein calculating the resolution index comprises computing a ratio between the respective measures of variability for the first and second populations of data and the separation distance.

8. The method according to claim 7, wherein the ratio is computed according to Equation A:
$$TaylorIndex = {TI}_{Clust 01 vsClust 02} = \frac{{\overline{X}}_{clust 01} - {\overline{X}}_{clust 02}}{{SD}_{clust 01} + {SD}_{clust 02}} \qquad ( A )$$
wherein:
x̅_{clust01} is the mean centroid position of the first population of data;
x̅_{clust02} is the mean centroid position of the second population of data;
SD_{clust01} is the standard deviation of the first population of data; and
SD_{clust02} is the standard deviation of the second population of data.

9. The method according to claim 1, wherein a resolution index is calculated for any given number of adjacent pairs of first and second populations of data.

10-11. (canceled)

12. The method according to claim 1, wherein the method further comprises computing Hartigan's dip statistic for the given number of adjacent pairs of first and second populations of data.

13. The method according to claim 1, wherein the method further comprises generating an image.

14. The method according to claim 13, wherein generating an image comprises compiling a heatmap of the resolution indices calculated for the given number of adjacent pairs of first and second populations of data.

15. The method according to claim 13, wherein generating an image further comprises compiling a heatmap of the Hartigan's dip statistics calculated for the given number of adjacent pairs of first and second populations of data.

16. The method according to claim 13, wherein generating an image further comprises plotting the populations of data on a scatter plot.

17. The method according to claim 1, wherein the data is comprised of signals from any given number of different parameters.

18. The method according to claim 1, wherein the method further comprises maximizing resolution between populations of data.

19. The method according to claim 18, wherein maximizing resolution between populations of data comprises computing a resolution score that provides a measure of separation between the different populations across the given number of different parameters of data.

20. The method according to claim 19, wherein the resolution score is computed according to Equation B and Equation C:
$$\begin{matrix}TaylorFactor = TF = \frac{\sqrt{m}}{n \cdot p} 100 & ( B ) \\ TaylorScore = log [ {\sum }_{x = ClustPair}^{n choose 2} TI ( x ) ] \cdot {TF}^{AdjustmentFactor} & ( C )\end{matrix}$$
wherein:
TI is a resolution index;
m is the number of cells;
n is the number of populations;
p is the number of parameters; and
AdjustmentFactor is a constant.

21-23. (canceled)

24. A system comprising:
an apparatus configured to produce data by analyzing a biological sample; and
a processor comprising memory operably coupled to the processor wherein the memory comprises instructions stored thereon, which when executed by the processor, cause the processor to:
obtain measures of variability for first and second populations of data, respectively;
determine a separation distance for the first and second populations of data from the obtained measures of variability; and
calculate a resolution index of the first and second populations of data by comparing their respective measures of variability with the separation distance.

25. The method according to claim 24, wherein the data is flow cytometer data.

26-69. (canceled)
